(19) United States
(12) Patent Application Publication
(10) Pub. No.: US 2025/0000867 A1
(43) Pub. Date: Jan. 2, 2025

(54) Compositions and Methods for the Prevention and Treatment of Hearing Loss

(71) Applicant: Ting Therapeutics, Inc, San Diego, CA (US)

(72) Inventor: Tal Teitz, Carlsbad, CA (US)

(73) Assignee: Ting Therapeutics, Inc, San Diego, CA (US)

(21) Appl. No.: 18/823,927

(22) Filed: Sep. 4, 2024

Related U.S. Application Data
(63) Continuation-in-part of application No. 18/676,439, filed on May 28, 2024.
Application No. 18/676,439 is a continuation-in-part of application No. 17/736,330, filed on May 4, 2022.
Application No. 17/736,330 is a division of application No. 17/580,755, filed on Jan. 21, 2022, now Pat. No. 11,433,073.
(60) Provisional application No. 62/947,059, filed on Dec. 12, 2019.

(51) Int. Cl. A61K 31/519; A61K 31/437; A61K 31/4439; A61K 31/4709; A61K 31/506; A61P 27/16

(52) U.S. Cl. CPC A61K 31/519; A61K 31/437; A61K 31/4439; A61K 31/4709; A61K 31/506; A61P 27/16

(57) ABSTRACT

Method, kit, and pharmaceutical compositions using an inhibitor of EGFR signaling for prevention or treatment of hearing loss are described as Dabrafenib is a therapeutic candidate for preventing cisplatin-induced hearing loss. It has a low effective dose of one tenth of the human equivalent dose (3 mg/kg administered twice day), a good toxicity profile, a therapeutic index of at least 25 in the multi-dose cisplatin regimen, protects both female and male mice, reduces hearing loss in two different strains of mice (FVB/NJ and CBA/CaJ), offers protection from weight loss that occurs during cisplatin chemotherapy, and persistence of hearing protection for at least four months after cisplatin treatments.

Statement regarding Federally Sponsored Research or development: This invention was made with government support under Grant Numbers DC006471 DC015010, DC015444, DC013879, DC013232, DC018850 and CA021765 awarded by the National Institutes of Health and Grant Numbers N00014-09-V-1014, N00014-12-V-0191, N00014-12-V-0775, awarded by the National Institute of Health and N00014-16-V-2315 awarded by the Office of Naval Research. The government has certain rights in the invention.

### THE BACKGROUND OF THE INVENTION

Field of the Invention: The invention provides a method for the treatment or prevention of hearing loss by administering to an animal in need thereof an inhibitor of epidermal growth factor receptor (EGFR) signaling. BRAF, MEK1/2, and ERK1/2 inhibition all protect from noise induced hair cell loss while BRAF and MEK1/2 inhibition protect from cisplatin ototoxicity.

### DESCRIPTION OF THE RELATED ART INCLUDING INFORMATION DISCLOSED UNDER 37 CFR 1.97 and 37 CFR 1.98

The ear is a complex organ composed of a labyrinth of structures responsible for hearing and balance. Perception of both hearing and balance lies in the ability of inner ear structures to transform mechanical stimuli to impulses recognized by the brain. The sensory receptors responsible for hearing are located in the cochlea, a spiral-shaped canal filled with fluid. Within the cochlea is the organ of Corti, which is lined with columnar sensory hair cells bridging the basilar membrane and the tectorial membrane. As sound waves pass through the organ of Corti, the basilar membrane vibrates causing the hair cells to bend back and forth. The movement depolarizes the hair cell, leading to release of neurotransmitters to the auditory nerve, which carries the impulse to the brain.

Cisplatin is a highly effective and commonly used chemotherapy agent for the treatment of a variety of cancers, but 40-60% of patients treated with cisplatin have irreversible hearing loss. Cisplatin negatively affects high frequency hearing more than lower frequencies primarily due to death of outer hair cells (OHCs) in the cochlear basal turn. Hair cells are the most common cochlear cell type to be affected by cisplatin but cells of the stria vascularis, spiral ganglion neurons, and supporting cells have also been reported to suffer deleterious effects. Cisplatin-induced hearing loss negatively impacts an individual's quality of life, leading to depression and social isolation, and impeding the development of language skills in young children treated with cisplatin. There is a clinical need to develop drugs that can protect from this highly common side effect of cisplatin treatment.

Currently, there is only one Food and Drug Administration (FDA)-approved drug for the treatment of cisplatin ototoxicity which has limited application. Sodium thiosulfate (STS) was recently approved by the FDA to reduce the risk of cisplatin-induced ototoxicity in pediatric patients 1 month or older with localized, nonmetastatic solid tumors, and represents a significant advancement in the field of hearing loss prevention. STS is administered to patients 6 hours after cisplatin treatment due to concerns over its interference with cisplatin's tumor killing efficacy even though no conclusive data demonstrates direct interference and no difference in hearing outcomes is observed with the delay in treatment. Recently, the antioxidant N-acetylcysteine (NAC) was shown to be otoprotective in phase-1 clinical trial in children and adolescents diagnosed with localized, nonmetastatic, cisplatin treated tumors. No severe adverse events occurred following N-acetylcysteine treatment which makes it a promising compound for the treatment of cisplatin-induced hearing loss. While the approval of Sodium thiosulfate and the clinical testing of N-acetylcysteine is beneficial for the treatment of cisplatin-induced hearing loss for localized solid tumor pediatric patients, there remains a clear therapeutic need to develop additional drugs that can protect from cisplatin ototoxicity for adults and children who do not meet the requirements for Sodium thiosulfate treatment such as patients with metastatic disease.

### SUMMARY OF THE INVENTION

The inventive subject matter includes a method to prevent ototoxicity comprising orally administering to subject in need thereof: a sufficient amount of a RAF inhibitor to prevent hearing loss, a sufficient amount of a ERK1/2 inhibitor to prevent hearing loss and a sufficient amount of a MEK1/2 inhibitor to prevent hearing loss.

The inventive subject matter further includes a method to prevent cisplatin-induced hearing loss including the steps of orally administering to subject in need thereof a pharmaceutical composition comprised of a sufficient amount of trametinib to prevent hearing loss due to cisplatin treatment, wherein the sufficient amount of trametinib, wherein the sufficient amount of trametinib ranges from 0.01 μM to 1.0 μM of trametinib.

The inventive subject matter further including the steps of orally administering to subject in need thereof after at least one rest period a pharmaceutical composition made of: a sufficient amount of trametinib to prevent hearing loss due to cisplatin treatment, wherein the sufficient amount of trametinib, wherein the sufficient amount of trametinib ranges from 0.01 μM to 1.0 μM of trametinib.

The inventive subject matter further includes a pharmaceutical composition in a unit dose form comprising cisplatin and an effective amount of a MEK1/2 inhibitor to prevent hearing loss due to ototoxicity, wherein the MEK inhibitor is trametinib. The pharmaceutical composition can also include a sufficient amount of RAF inhibitor to prevent hearing loss due to ototoxicity. The pharmaceutical composition can also include a sufficient amount of ERK inhibitor to prevent hearing loss due to ototoxicity.

Brief Description of the Drawings: The patent or application file contains at least one drawing executed in color. Copies of this patent or patent application publication with color drawing(s) will be provided by the Office upon request and payment of the necessary fee.

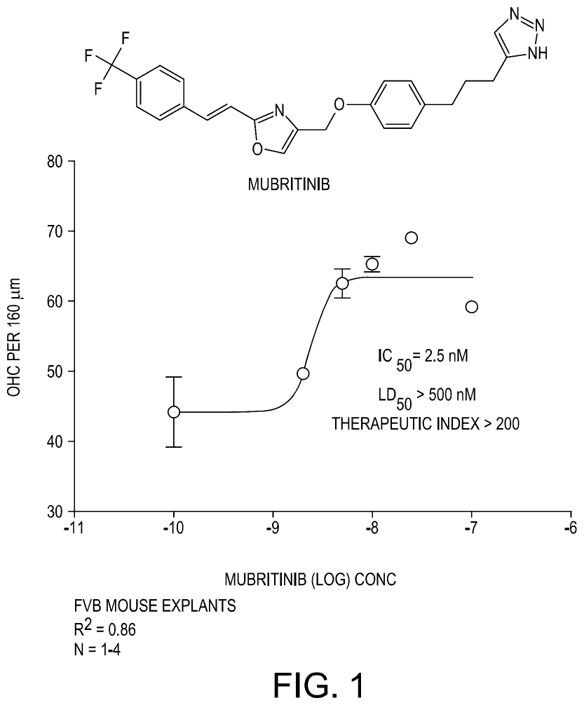

FIG. 1 shows that the EGFR inhibitor MUBRITINIB (whose structure is shown) protects against cisplatin-induced hair cell loss in mouse cochlear explants with IC₅₀ of 2.5 nM and LD₅₀ of >500 nM (Therapeutic Index of >200). Number of explants: 1-4 at each dose; FVB mouse cochlear explants were treated with 150 μM cisplatin and cochlear middle turns were analyzed; curve fitting with R² of 0.86. Note that IC₅₀ values of MUBRITINIB were consistent in all assays (HEI-OC1 cells and explants) demonstrating its specificity and potency.

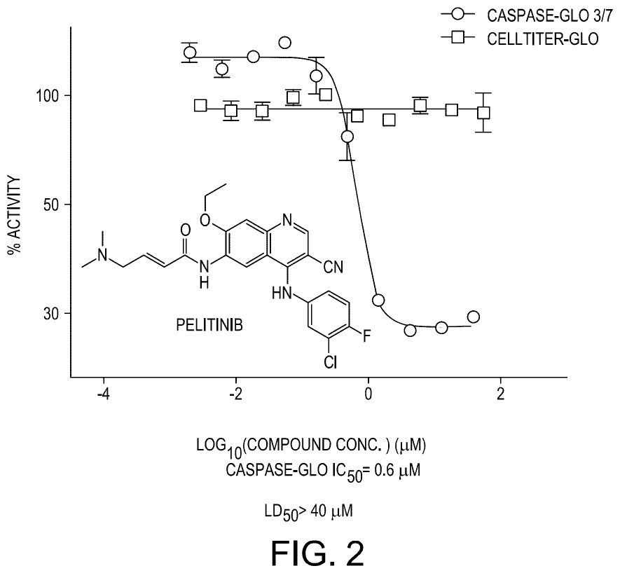

FIG. 2 shows that the EGFR inhibitor Pelitinib (whose structure is shown) protects against cisplatin-induced hair cell loss. Pelitinib is an irreversible inhibitor of EGFR that exhibits protective effects against cisplatin-induced Caspase-3/7 activity in HEI-OC1 cells with IC₅₀ of 0.6 μM (cisplatin-Caspase-Glo 3/7) and LD₅₀ of >40 μM (CELLTITER-GLO).

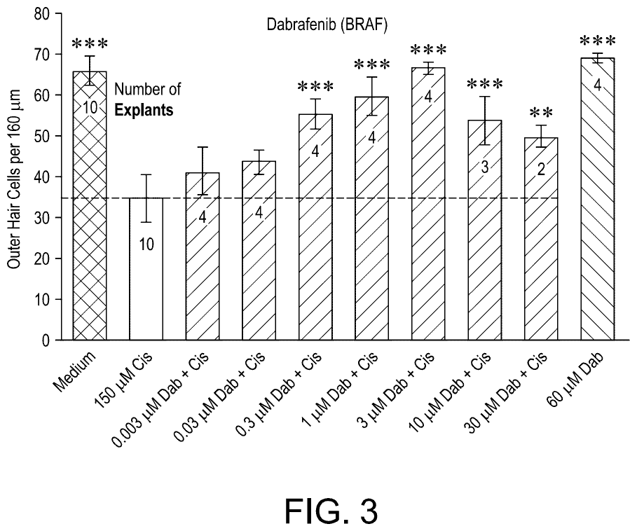

FIG. 3 shows dose-response of dabrafenib (Dab) in mouse cochlear explants treated with or without cisplatin. Dab alone or Dab added 1 h before cisplatin (150 μM) to P3 FVB cochlear explants for 24 h. Number of explants for each dose of Dab was shown. ***:P<0.001; **:P<0.01; *:P<0.05 (Student's T-test) comparing to Cis alone.

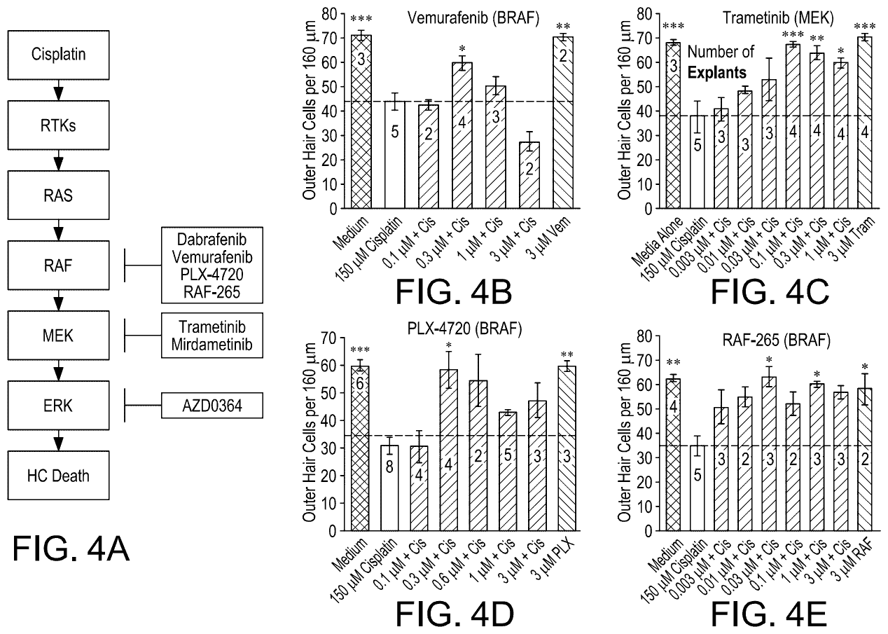

FIG. 4A shows signaling cascade of RTKs (receptor tyrosine kinases), RAS, RAF, MEK, and ERK in cisplatin-induced hair cell death and current small molecule inhibitors for RAF, MEK and ERK in otoprotection.

FIG. 4B shows the activity of B-Raf inhibitor Vemurafenib to protect in the cochlear explant culture assay against cisplatin-induced hair cell death. Dose-responses of the compounds in P3 FVB mouse cochlear explants treated with or without cisplatin were shown. See FIG. 3 for the meaning of other labels.

FIG. 4C shows the activity of B-Raf inhibitor Trametinib.

FIG. 4D shows the activity of B-Raf inhibitor PLX-4720.

FIG. 4E shows the activity of B-Raf inhibitor RAF-265.

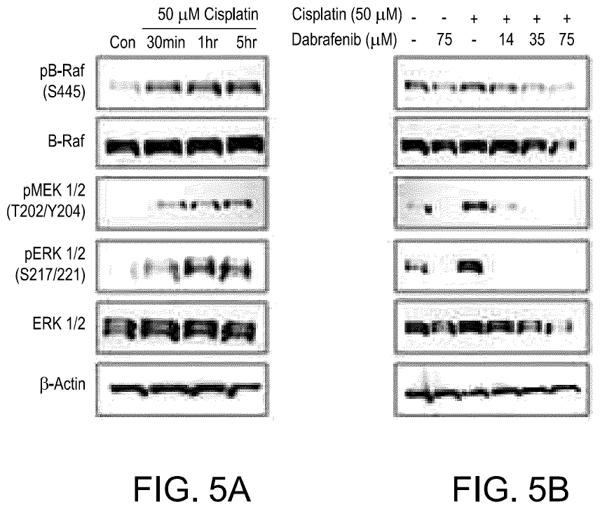

FIG. 5A shows Dabrafenib mitigates cisplatin activated B-Raf signaling cascade in HEI-OC1 cells. Western blots show representative changes of each signaling molecule upon cisplatin and Dab treatment at specific time.

FIG. 5B shows Dabrafenib mitigates cisplatin activated B-Raf signaling cascade in HEI-OC1 cells. Western blots show representative changes of each signaling molecule upon cisplatin and Dab treatment at specific doses (5B)

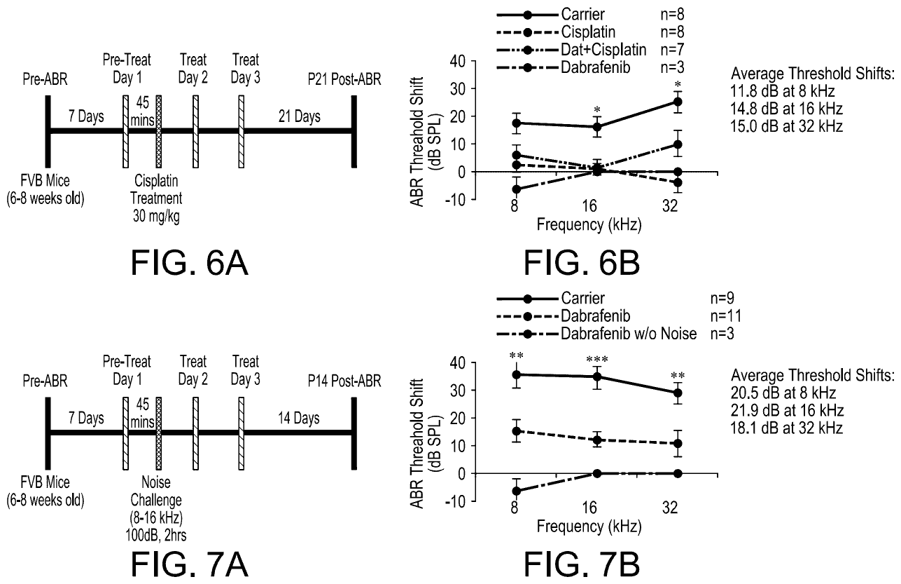

FIG. 6A shows schedule of administration of dabrafenib (100 mg/kg) and cisplatin (30 mg/kg) to adult FVB mice (males and females).

FIG. 6B shows reduced ABR threshold shifts of 11.8-15.0 dB in average were recorded on day 21 after first day of cisplatin (30 mg/kg) and dabrafenib (100 mg/kg) co-treatment, mean±SEM, *, P<0.05, compared to cisplatin alone by two-way ANOVA followed by a Bonferroni comparison.

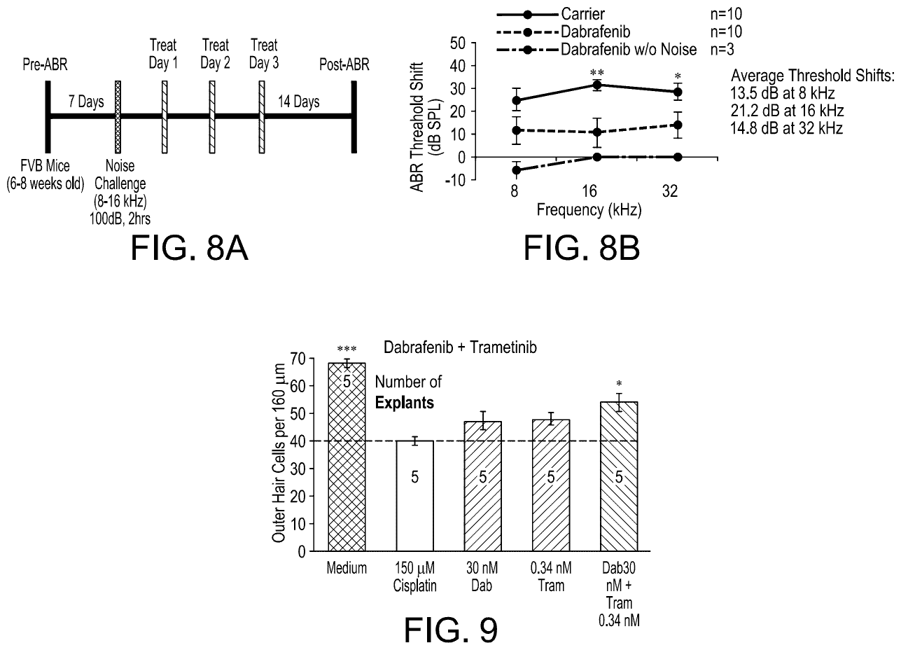

FIG. 7A shows schedule of administration of dabrafenib (100 mg/kg) and noise exposure to adult FVB mice (males and females).

FIG. 7B shows reduced ABR threshold shifts of 18.1-21.9 dB in average were recorded on day 14 after first day of dabrafenib (100 mg/kg) and noise exposure, mean±SEM, **, P<0.01, ***, P<0.001, compared to carrier by two-way ANOVA followed by a Bonferroni comparison.

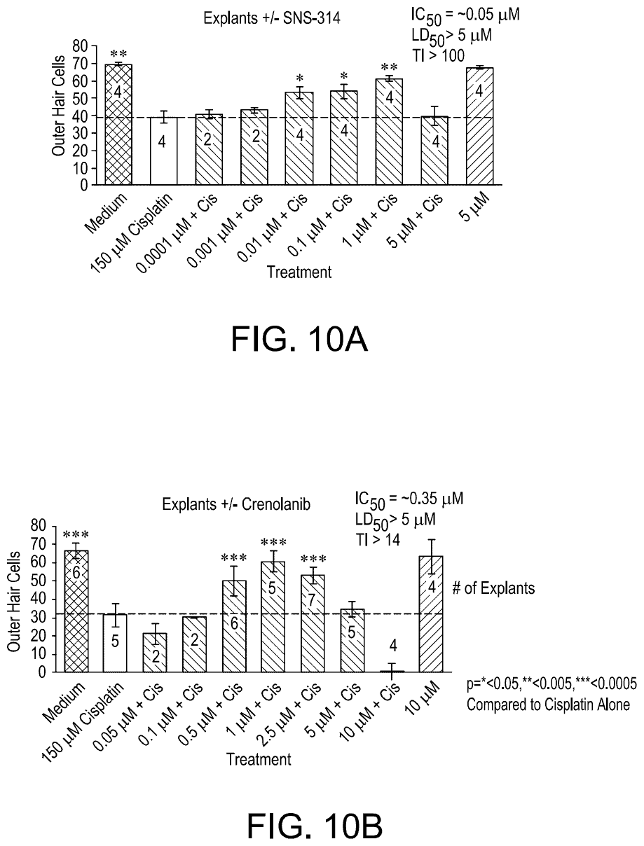

FIG. 8A shows schedule of administration of dabrafenib (60 mg/kg ×2 daily) and noise exposure to adult FVB mice (males and females).

FIG. 8B shows reduced ABR threshold shifts of 13.5-21.2 dB on average were recorded on day 14 after first day of dabrafenib (60 mg/kg ×2 daily) and noise exposure, mean±SEM, **, P<0.01, ***, P<0.001, compared to carrier by two-way ANOVA followed by a Bonferroni comparison.

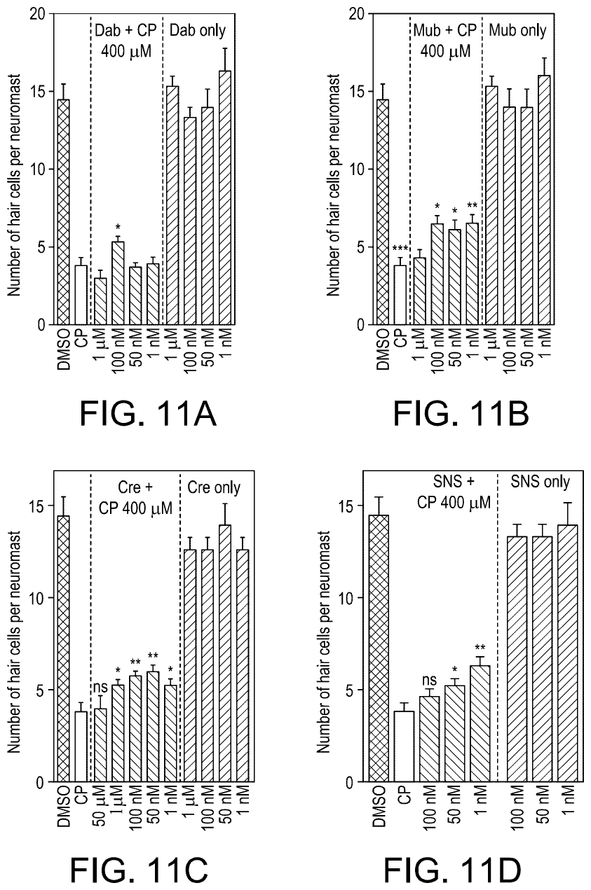

FIG. 9 shows Testing a B-Raf/MEK1/2 inhibitor combination in mouse cochlear explant cultures. Compounds alone or combination of the compounds were added 1 h before cisplatin (150 μM) to P3 FVB cochlear explants for 24 h, and number of outer hair cells per 160 μm of middle turn regions of the cochlea were counted by phalloidin staining, mean±SEM, P=*<0.05, P=***<0.001, compared to cisplatin alone by unpaired two-tailed Student's t-test. The initial molar ratio between the compounds tested was determined by the ratio given currently to cancer patients (dabrafenib at 150 mg twice daily plus trametinib at 2 mg once daily).

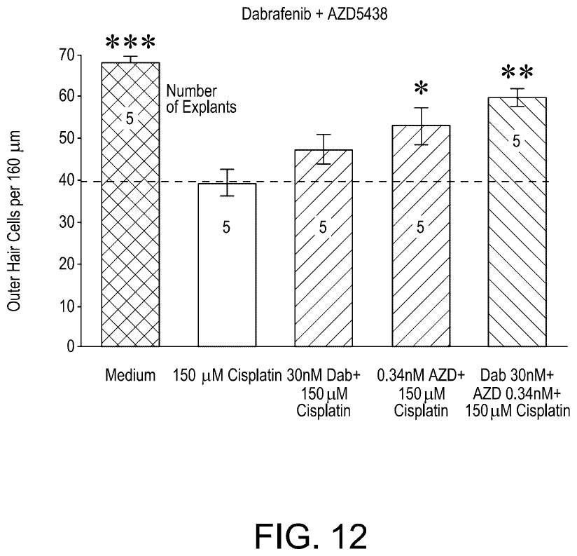

FIG. 10A shows data from mouse cochlear explants for a number of inhibitors (Mubritinib, SNS-314, and Crenolanib).

FIG. 10B shows data from mouse cochlear explants for a number of inhibitors (Mubritinib, SNS-314, and Crenolanib).

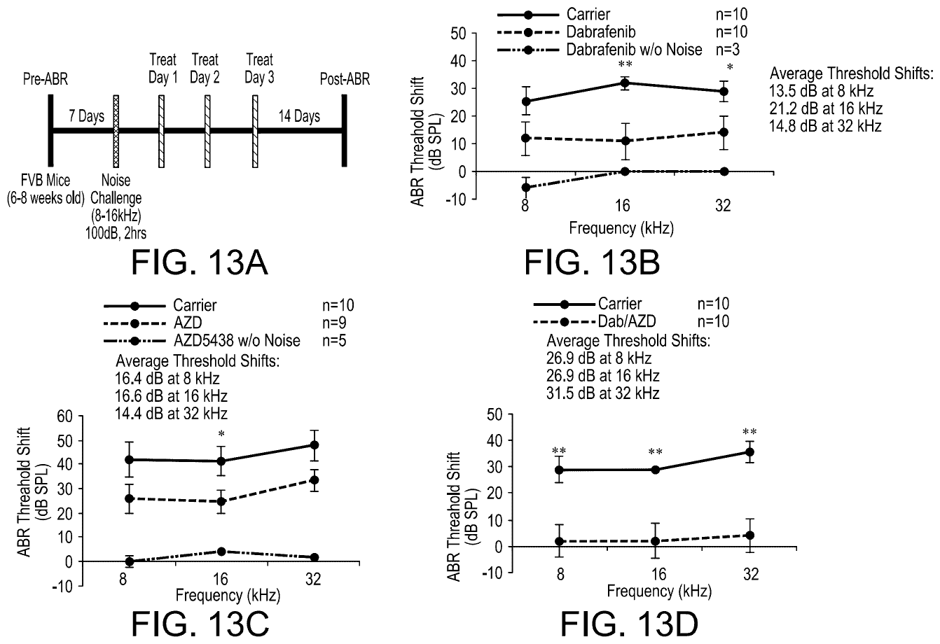

FIG. 11A, shows compounds protective effect in Zebrafish (Dabrafenib), Lateral line neuromasts of zebrafish were stained and number of hair cells per neuromast were counted. Cisplatin (CP): 400 μM. *, **, and ***:P<0.05, 0.01, and 0.001 compared to CP alone.

FIG. 11B shows compounds protective effect in Zebrafish (Mubritinib) Lateral line neuromasts of zebrafish were stained and number of hair cells per neuromast were counted. Cisplatin (CP): 400 μM. *, **, and ***:P<0.05, 0.01, and 0.001 compared to CP alone.

FIG. 11C shows compounds protective effect in Zebrafish (Crenolanib). Lateral line neuromasts of zebrafish were stained and the number of hair cells per neuromast were counted. Cisplatin (CP): 400 μM. *, **, and ***:P<0.05, 0.01, and 0.001 compared to CP alone.

FIG. 11D shows compounds protective effect in Zebrafish (SNS-314). Lateral line neuromasts of zebrafish were stained and the number of hair cells per neuromast were counted. Cisplatin (CP): 400 μM. *, **, and ***:P<0.05, 0.01, and 0.001 compared to CP alone.

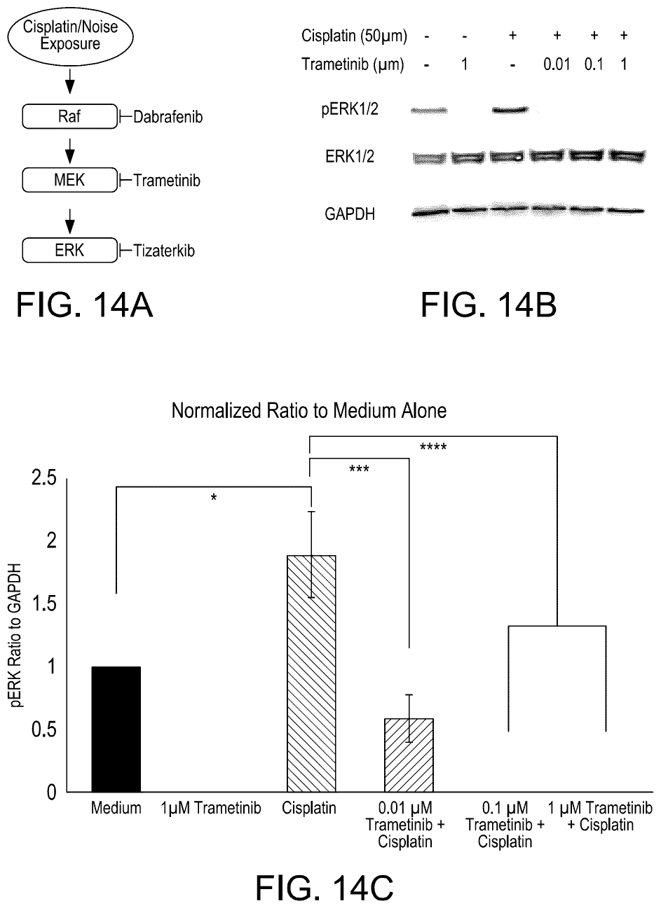

FIG. 12 shows the compounds Dabrafenib (a B-Raf kinase inhibitor, 30 nM) and AZD5438 (a CDK2 kinase inhibitor, 0.34 nM) protects against cisplatin, better than individual inhibitor, in mouse cochlear explants.

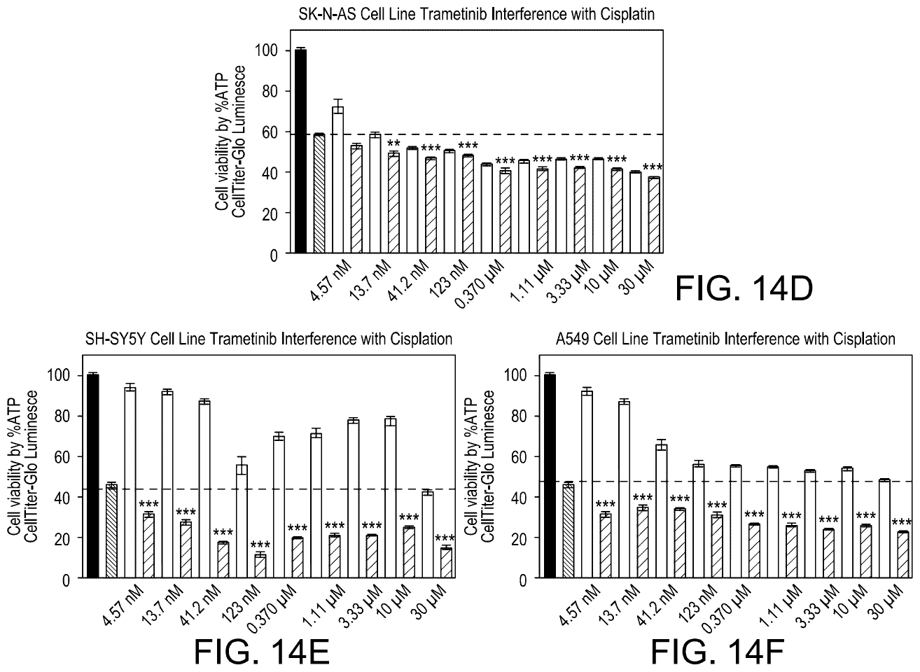

FIG. 13A shows protection against noise injury in mice by oral delivery of a combination of inhibitors (Dabrafenib 60 mg/kg ×2 daily, AZD5438 35 mg/kg ×2 daily).

FIG. 13B shows protection against noise injury in mice by oral delivery of a combination of inhibitors (Dabrafenib 60 mg/kg ×2 daily, AZD5438 35 mg/kg ×2 daily).

FIG. 13C shows protection against noise injury in mice by oral delivery of a combination of inhibitors (Dabrafenib 60 mg/kg ×2 daily, AZD5438 35 mg/kg ×2 daily).

FIG. 13D shows protection against noise injury in mice by oral delivery of a combination of inhibitors (Dabrafenib 60 mg/kg ×2 daily, AZD5438 35 mg/kg ×2 daily).

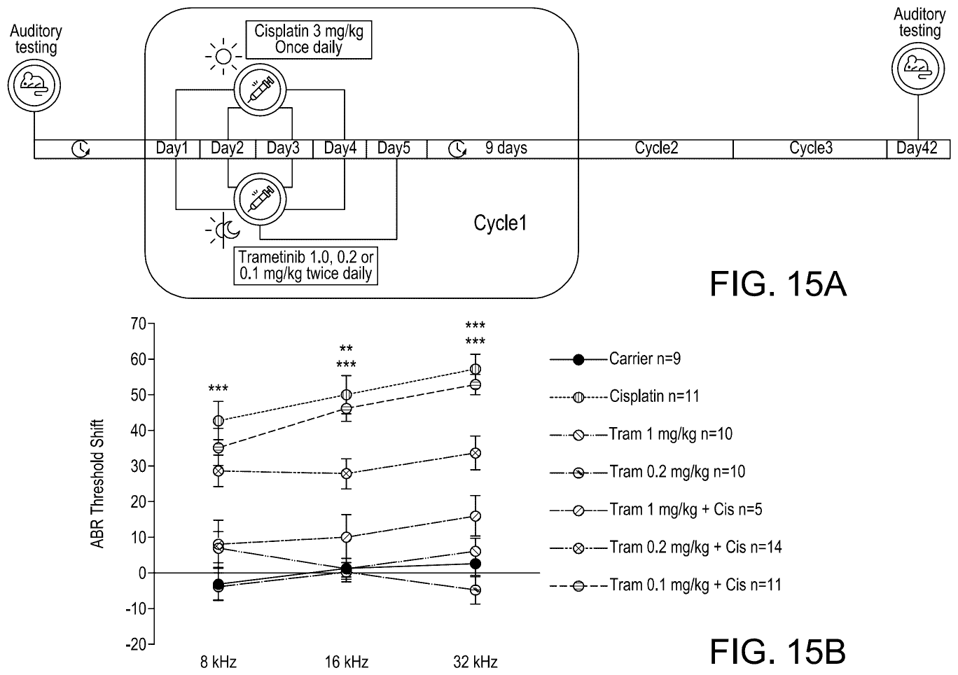

FIG. 14A shows a schematic of the MAPK phosphorylation cascade in which dabrafenib inhibits BRAF, trametinib inhibits MEK1/2, and tizaterkib inhibits ERK1/2.

FIG. 14B shows representative western blots of HEI-OC1 cell lysates treated with medium, cisplatin, and different concentrations of trametinib. Treatment groups from left to right are as follows: medium alone, 1 μM trametinib alone, 50 μM cisplatin alone, 50 μM cisplatin+0.01 μM trametinib, 50 μM cisplatin+0.1 μM trametinib, and 50 μM cisplatin+1 μM trametinib.

FIG. 14C shows quantification of western blots represented in FIG. 14A. A total of four separate experiments were performed. The ratio of pERK to GAPDH was measured for each individual lane and all groups were then normalized to the medium alone treatment group. Data shown as means±SEM. * P<0.05, *** P<0.001, **** P<0.0001. All groups compared to one another by one-way ANOVA with Bonferroni post hoc test.

FIG. 14D shows percentage of cell viability for SK-N-AS cell line treated with cisplatin and various concentrations of trametinib starting at 4.57 nM and going up to 30 μM in increments of 3-fold increases.

FIG. 14E shows percentage of cell viability for SH-SY5Y cell line treated with cisplatin and various concentrations of trametinib as mentioned in FIG. 14D.

FIG. 14F shows percentage of cell viability for A549 cell line treated with cisplatin and various concentrations of trametinib as mentioned in FIG. 14D). Medium alone, Cisplatin alone, trametinib alone, and trametinib+cisplatin. All wells treated with cisplatin had the same concentration of cisplatin and increasing concentrations of trametinib were used starting at 4.57 nM going up to 30 μM going from left to right. All treatments were normalized to medium alone treated cells and compared to the cisplatin alone treatment. Data shown as mean±SEM *P<0.05, ** P<0.01, *** P<0.001 compared to cisplatin alone by one-way ANOVA with Bonferroni post.

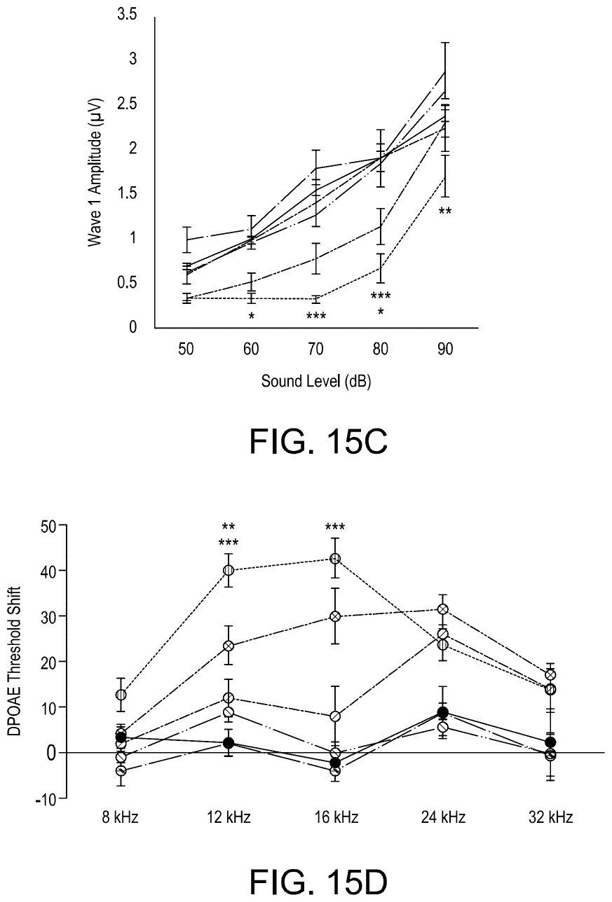

FIG. 15A shows a Schedule of administration of dabrafenib and cisplatin in a translational, multi-cycle cisplatin treatment protocol using CBA/CaJ mice. Each cycle consisted of four days of treatment with 3 mg/kg cisplatin in the morning and five days of treatment with 1 or 0.2 mg/kg trametinib in the morning and evening. A 9-day recovery period followed the 5 days of treatment. This cycle was repeated a total of 3 times. Auditory testing occurred before treatment began and immediately after cycle 3 (day 42).

FIG. 15B shows ABR threshold shifts recorded immediately after the completion of cycle 3 (day 42) in protocol shown in FIG. 15A.

FIG. 15C shows amplitudes of ABR wave 1 at 16 kHz from FIG. 15B.

FIG. 15D shows DPOAE threshold shifts recorded after the completion of cycle 3 (day 42) in protocol shown in (A). Carrier alone, 1 mg/kg trametinib alone, 0.2 mg/kg trametinib alone, cisplatin alone, 1 mg/kg trametinib co-administered with cisplatin, 0.2 mg/kg trametinib co-administered with cisplatin 0.1 mg/kg trametinib co-administered with cisplatin. Data shown as means±SEM, *P<0.05, ** P<0.01, *** P<0.001 compared to cisplatin alone by two-way ANOVA with Bonferroni post hoc test.

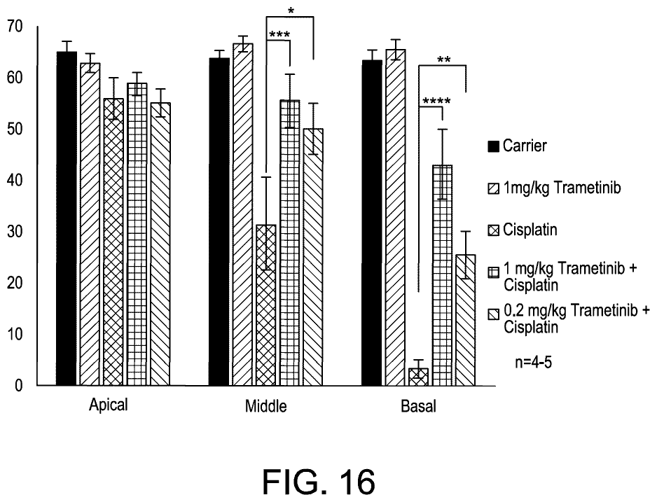

FIG. 16 shows quantification of the number of outer hair cells per 160 μm per section for apical turn, middle turn, and basal turn of cochlea. Carrier alone, 1 mg/kg trametinib alone, cisplatin alone, 1 mg/kg trametinib co-administered cisplatin, and 0.2 mg/kg trametinib co-administered cisplatin (green). Data shown as means±SEM, *P<0.05, ** P<0.01, *** P<0.001, **** P<0.0001 compared to cisplatin alone by two-way ANOVA with Bonferroni post hoc test. n=4-5.

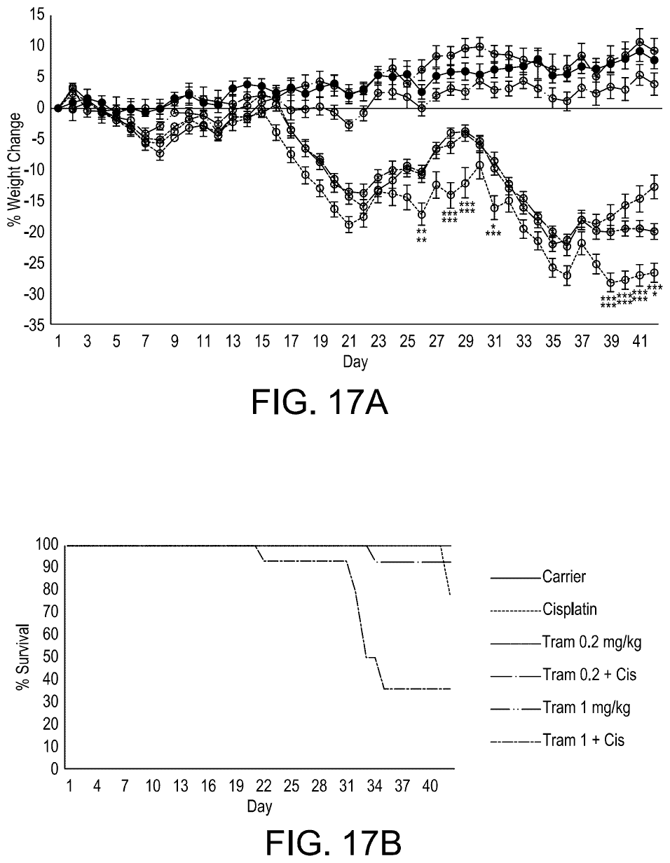

FIG. 17A shows weight loss over the 42-day treatment protocol shown in FIG. 2A. Data shown as means±SEM, *P<0.05, ** P<0.01, *** P<0.001 compared to cisplatin alone by two-way ANOVA with Bonferroni post hoc test.

FIG. 17B shows Kaplan-Meier survival curves of mouse cohorts going to day 42 following protocol in FIG. 15A. Carrier alone (black), 1 mg/kg trametinib alone, 0.2 mg/kg trametinib alone cisplatin alone, 1 mg/kg trametinib co-administered cisplatin, and 0.2 mg/kg trametinib co-administered cisplatin.

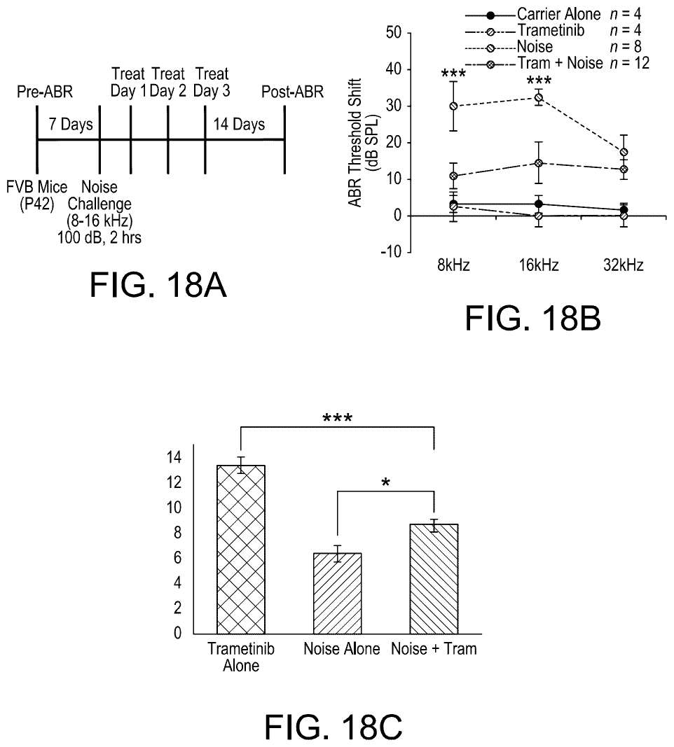

FIG. 18A shows noise exposure and treatment protocol. ABR pre-hearing tests were performed and then mice were exposed to 100 db SPL noise for 2 hours. Starting 24 hours after noise exposure, mice were treated with 3.15 mg/kg trametinib twice a day for 3 total days, once in the morning and once at night. 14 days after noise exposure, ABR hearing tests were performed again to determine the hearing loss for each mouse.

FIG. 18B shows ABR threshold shifts from the treatment protocol shown in (A). Carrier alone, trametinib alone noise alone, and trametinib+noise. Data shown as means±SEM, *** P<0.001 compared to cisplatin alone by two-way ANOVA with Bonferroni post hoc test.

FIG. 18C shows Quantification of the average number of ctbp2 puncta per IHC for each treatment group. Trametinib alone, noise alone, and noise+trametinib. Data shown as means±SEM, *P<0.05, *** P<0.001 with all groups compared to one another by one-way ANOVA with Bonferroni post hoc test.

### DETAILED DESCRIPTION OF THE INVENTION

### Definitions

As used herein, the singular forms “a,” “an” and “the” include plural referents unless the content clearly dictates otherwise.

As used herein, the terms “about” and “approximately,” when used to modify a numeric value (e.g., “about 80 mg”) or one or more amounts specified in a range of numeric values (e.g., “about 80 mg to about 400 mg”), indicate the numeric value and functionally equivalent values, and reasonable deviations from the value known to or understood by those in the art, including persons of ordinary skill in the art or those skilled in the art. For example, values within +10% or +5% are within the intended meaning of a recited value. As such, “about 80 mg” is understood to encompass from “76 mg to 84 mg” or from “72 mg to 88 mg” as if written out each time. Thus, as used herein, the terms “about” and “approximately” intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art.

As used herein, the terms “administering”, and “administration” refer to any method of providing a compound or composition to a subject. In some embodiments, the compound or composition is a pharmaceutical preparation. Such methods include, but are not limited to, oral administration, auricular administration, transdermal administration, administration by inhalation, nasal administration, topical administration, intraaural administration, intradural administration, intracerebral administration, sublingual administration and parenteral administration, including injectable such as intravenous administration, intra-arterial administration, intramuscular administration, and subcutaneous administration. Administration can be one-time, intermittent, or continuous. In some embodiments, a compound or composition of the invention is administered therapeutically, e.g., to treat an existing, diagnosed or suspected disease, disorder or condition. In some embodiments, a compound or composition of the invention is administered prophylactically, e.g., administered to prevent or inhibit progression of a disease, disorder or condition, or prevent the spread of a disease, disorder or condition.

As used herein, “effective amount” or “therapeutically effective amount” refers to an amount effective, at dosages and for periods of time as described in the dose regimens herein, to achieve a desired result in treatment of a disease, disorder, defect or condition for which one or more compounds or compositions of the invention are administered to a subject or to a patient. For example, and not by way of limitation, a “therapeutically effective amount” can refer to an amount of a cytoprotective drug compound or composition (e.g. a compound or composition comprising or consisting essentially of a cytoprotective drug), including but not limited to those disclosed herein, that is able to treat a disease, defect, disorder or condition when administered in accordance with the invention.

In some embodiments, “effective amount” or “therapeutically effective amount” refers to an amount effective a compound or composition, at dosages and for periods of time as described in the dose regimens herein or used by those in accordance with methods of the invention, to achieve a desired result or results in the treatment of one or more signs and/or symptoms of cisplatin-induced or noise-induced hearing loss. Symptoms include those described or referenced herein, for example, loss of hearing sensitivity, ringing in the ear, inner ear pain, and inner ear swelling. Therapeutically effective amounts include an amount that is sufficient to achieve at least one desired therapeutic result, or to have at least one effect on at least one undesired symptom associated with a disease, disorder, defect or condition, including but not limited to those referenced herein. The specific therapeutically effective dose level for any particular patient may be varied or depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors used in the medical arts in determining dose levels. For example, those in the art may start doses of a compound or composition at levels lower than those required to achieve the desired therapeutic effect and to gradually increase the dosage until the desired effect is achieved. In some embodiments, the result of treatment with an “effective amount” provides a durable result following treatment. In some embodiments, treatment with an effective amount is discontinued following a desired result. In some embodiments, treatment with an effective amount is continued following a desired result. In some embodiments, treatment with an effective amount is paused and then continued following a desired result. The doses disclosed herein are therapeutically effective amounts; however, the methods are not limited to those doses or dose amounts and include the use of other therapeutically effective amounts.

In some embodiments, a compound, composition or preparation can be administered in a “prophylactically effective amount”; that is, an amount effective for prevention of cisplatin-induced or noise-induced hearing loss. The compounds and compositions herein can be used to prevent, inhibit, or reduce the occurrence of one or more signs or symptoms of noise-induced or cisplatin-induced hearing loss.

A “maintenance dose” or a “preventative dose” is a dose intended to be a therapeutically effective or a prophylactically effective dose. In some embodiments, the maintenance doses may be therapeutically effective doses and others may be sub-therapeutic doses. This disclosure includes dosing regimens and oral dosage formulations for the administration of maintenance doses of cytoprotective drugs. Such doses of cytoprotective drugs can be administered in cycles (e.g., a “first cycle”, a “second cycle”, a “third cycle”, etc.), during which each cycle a different amount of the cytoprotective drug is administered to a subject. For example, in some embodiments, the invention features a method of treating one or more signs and/or symptoms of noise-induced or cisplatin-induced hearing loss in a subject by (i) administering a loading-dose of a cytoprotective drug to the subject; and (ii) administering one or more maintenance doses of the cytoprotective drug to the subject, wherein each of the loading-dose and the maintenance doses are administered in an amount that together are sufficient to treat the one or more signs and/or symptoms of cisplatin-induced or noise-induced hearing loss. For example, the loading dose can be administered by injection (e.g., subcutaneously) or orally followed by maintenance dosing administered orally, intravenously, nasally, subcutaneously, transdermally, or by direct administration to the inner ear. In some embodiments, the loading-dose is administered orally. In some embodiments, the loading-dose is administered by direct administration to the inner ear by the methods described herein. In some embodiments, the introductory or loading dose is about 15 to 30 mg/kg administered daily and the maintenance dose is about 0.6 to about 15 mg/kg administered about once a week. In some embodiments, the dose for the induction phase, e.g., the introductory or loading dose, is a dose of 15 to 30 mg/kg administered at least once per day and the dose for the maintenance phase is about 0.6 to 15 mg/kg once a week.

The term “pharmaceutical formulation” refers to a preparation which is in such form as to permit the biological activity of an active ingredient contained therein to be effective, and which does not contain additional components that are unacceptably toxic to a subject to which the formulation would be administered. A “pharmaceutical composition” refers to a mixture of substances suitable for administering to a subject that includes one or more active ingredients or pharmaceutical agents (e.g., one or more cytoprotective drugs). For example, a pharmaceutical composition may include one or more compounds of the invention and a sterile aqueous solution or a pharmaceutically acceptable carrier.

A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, a buffer, excipient, stabilizer, or preservative.

As used herein, the term “prevents” or “preventing” refers to precluding, averting, obviating, forestalling, stopping, or hindering something from happening. It is understood that where reduce, inhibit or prevent are used herein, unless specifically indicated otherwise, the use of the other two words is also expressly disclosed, and that they refer to reducing, inhibiting or preventing in whole or in part for the therapeutic and/or prophylactic purposes described herein.

As used herein, the term “subject” or the like, refers to any animal, including humans, domestic and farm animals, and zoo, wild animal park, sports, or pet animals, such as dogs, horses, cats, fish (e.g., zebrafish), sheep, pigs, cows, etc. In some instances, however, a patient may refer to a subject afflicted with specific a condition, disease, defect, syndrome or disorder. The terms “subject” and “patient” include human and veterinary subjects. The preferred animal herein is a human, including adults, children, and the elderly. The term “subject” does not denote a particular age or sex. In some embodiments, subjects may include animals used in scientific experiments (e.g. mice, rats, rabbits, sheep, goats, or other laboratory subjects). In other embodiments, the term “subjects” may exclude one or more or all animals used in scientific experiments, and a method may specify that it does not include one or more or all animals used in scientific experiments. In some embodiments, subjects may include non-animals and other things used in scientific experiments (e.g., fruit flies, cells, cell cultures, tissues, organs, 3D tissue culture (such as organs-on-a-chip). In other embodiments, the term “subjects” may exclude one or more or all non-animals or other things used in scientific experiments and a method may specify that is does not include one or more or all non-animals or other things used in scientific experiments. In some embodiments of the disclosed methods, a subject has been diagnosed with a need for treatment of one or more signs and/or symptoms associated with cisplatin-induced or noise-induced hearing loss.

As used herein, “treatment”, “treat” and “treating”, refers to an attempt to alter the course of the individual, tissue or cell being treated, and can be performed either for prophylaxis or during the course of clinical pathology. Thus, the term “treatment” includes the management or care of a subject, or of a patient, with the intent to cure, ameliorate, stabilize, or prevent a disease, disorder, defect or condition. This term includes active treatment, that is, treatment directed specifically toward the improvement of a disease, disorder, defect or condition, and also includes causal treatment, that is, treatment directed toward removal of the cause of the associated disease, disorder, defect or condition. In addition, this term includes palliative treatment, that is, treatment designed for the relief of symptoms rather than the curing of the disease, disorder, defect or condition; preventative treatment, that is, treatment directed to minimizing or partially or completely inhibiting the development of the associated disease, disorder, defect or condition; and supportive treatment, that is, treatment employed to supplement another specific therapy directed toward the improvement of the associated disease, disorder, defect or condition. In various aspects, the term covers any treatment of a subject, including a mammal (e.g., a human), and includes: (i) preventing a disease, disorder or condition from occurring in a subject that can be predisposed to the disease, disorder or condition but has not yet been diagnosed as having it; (ii) inhibiting a disease, disorder or condition, i.e., arresting or slowing its development; or (iii) relieving a disease, disorder or condition, i.e., causing regression of the disease, disorder or condition. In some embodiments, the subject is a human. Treatment does not necessarily imply that a subject or patient is treated until total recovery following treatment, or that treatment of a subject or patient results in total recovery. Accordingly, “treatment” may also include maintaining or promoting a complete or partial state of remission in a subject or patient, or an alleviation or relief from symptoms of the disease, disorder, defect of conditions being treated, whether in whole or in part, temporary or permanent. The term “preventing” means preventing in whole or in part or ameliorating or controlling. The term “ameliorate,” “ameliorating” and “amelioration” and the like refer an improvement in one or more signs or symptoms of noise-induced or cisplatin-induced hearing loss, a reduction in the severity of one or more signs or symptoms of noise-induced or cisplatin-induced hearing loss, or an inhibition of progression or worsening of one or more signs or symptoms of noise-induced or cisplatin-induced hearing loss or disorder.

In some embodiments, compounds, compositions, methods, and kits of the invention are used in the treatment of a subject or a patient for one or more signs and/or symptoms of noise-induced or cisplatin-induced hearing loss.

In some embodiments, compounds, compositions, methods, and kits of the invention are used alleviation of one or more signs or symptoms of, or diminishment of one or more direct or indirect pathological consequences of noise-induced or cisplatin-induced hearing loss.

Hearing loss is one of the most common types of disability; however, there is only one FDA-approved drug to prevent any type of hearing loss. Treatment with the highly effective chemotherapy agent, cisplatin, and exposure to high decibel noises are two of the most common causes of hearing loss. The mitogen activated protein kinase (MAPK) pathway, a phosphorylation cascade including RAF, MEK1/2, and ERK1/2, has been implicated in both types of hearing loss. Pharmacologically inhibiting BRAF or ERK1/2 is protective from noise and cisplatin-induced hearing loss in multiple mouse models.

Trametinib, a MEK1/2 inhibitor, protects from cisplatin induced outer hair cell death in mouse cochlear explants; inhibiting MEK1/2 has not yet been shown to be protective from hearing loss in vivo. Trametinib protects from cisplatin-induced hearing loss in a translationally relevant mouse model and does not interfere with cisplatin's tumor killing efficacy in cancer cell lines. Higher doses of trametinib were toxic to mice when combined with cisplatin but lower doses of the drug were protective from hearing loss without any known toxicity. Trametinib also protected mice from noise-induced hearing loss and synaptic damage. MEK1/2 inhibition protects from both insults of hearing loss and that targeting all three kinases in the MAPK pathway protects from cisplatin and noise-induced hearing loss in mice.

The widely used chemotherapy cisplatin causes permanent hearing loss in 40-60% of cancer patients. Human cancer patients typically receive a week of daily cisplatin infusions in cycles spaced a few weeks apart. Employing a clinically relevant cisplatin protocol and three 1:5 dilutions of the drug dabrafenib (15, 3, 0.6 mg/kg), we conclude that dabrafenib has an average protection of 19 dB at 8 kHz, 25 dB at 16 KHz, and 34 dB at 32 kHz, after cisplatin treatment with a low dose 3 mg/kg twice daily. Significantly, the dose of 3 mg/kg/bw dabrafenib, twice daily, was found to be as effective as the 15 mg/kg/bw and is approximately one tenth of the equivalent dabrafenib human dose given to cancer patients. 15 and 3 mg/kg dabrafenib exhibited the same hearing protection with no statistically significant difference between the groups. Thus, 3 mg/kg was determined to be the minimal effective dose in this model. The lowest dose tested of 0.6 mg/kg dabrafenib, which is equivalent to one fiftieth of the human equivalent dose, still demonstrated protection of 12 dB at 8 kHz, 15 dB at 16 kHz, and 20 dB at 32 kHz is still detected, yet it is not as effective as 3 or 15 mg/kg dabrafenib. The multi-dose protocol demonstrated a therapeutic window of at least 25 for dabrafenib in vivo. Protection was observed with a dose as high as 15 mg/kg and as low as 0.6 mg/kg. Higher doses of dabrafenib were not tested, however, previous data obtained from the single, high dose cisplatin protocol demonstrated 100 mg/kg dabrafenib daily was well tolerated. A wide therapeutic index is important for the clinical application of dabrafenib to give clinicians flexibility with dosage without toxicity to the patient.

Protection from weight-loss in the cisplatin and dabrafenib co-treated groups, employing either the single-dose protocol or the multi-dose regimen, is an unexpected and exciting phenomenon in our studies. Dabrafenib significantly reduces the weight loss typically seen in mice during cisplatin treatment and thus helps maintain the general well-being of the animals. At this stage, we do not know the molecular mechanism for the reduction in weight loss or whether it is involved in modulating the brain appetite pathways.

Toxicity of dabrafenib with cisplatin treatment was tested in this study in the kidney and livers of the treated animals. Combining two drugs together could pose some systemic toxicity issues; therefore, we wanted to ensure that the combination of dabrafenib and cisplatin was not toxic to major organs that can be damaged from cisplatin. These organs were chosen as it is known that, in addition to the ear, cisplatin accumulates and can cause damage in these tissues. No significant damage was recorded by H&E, PAS, and Masson's Trichrome staining in the kidneys or livers of the mice at days 42 and 165 with the co-treatments. Dabrafenib alone, being an FDA-approved drug, was not expected to cause significant damage to the kidneys and livers of the mice in the doses tested in this study, but the toxicity and ototoxicity of the co-treatments were unknown. This demonstration of no significant toxicity or ototoxicity of the drug co-treatments is vital for future clinical trials.

Cisplatin has been shown to accumulate in the inner ear by the Breglio et al. study and may cause long-term hearing loss and possible reduced protection when drug administration does not continue after the cessation of cisplatin treatment. For that reason, it is important to test if dabrafenib will protect not only on day 42 when the cisplatin cycles are completed, but also at longer time points, such as four months after the treatments. The data shows that dabrafenib co-treated mice still have significantly better hearing ability compared to cisplatin alone mice. The hearing protection is sustained for up to 4 months following the end of cisplatin treatment, which indicates the protection dabrafenib offers from cisplatin ototoxicity is stable and not acute. Mice only need to be treated with dabrafenib while cisplatin is administered and more treatments following the cessation of cisplatin are not required to confer protection. This limits the amount of drug patients would need to receive to get optimal hearing protection from dabrafenib.

It has now been found that inhibitors of EGFR and proteins downstream or associated therewith protect hair cells damaged by cisplatin, antibiotics, noise, aging or other ototoxic insults. Accordingly, this invention provides compositions and methods for the prevention and treatment of hearing loss using an inhibitor of EGFR. Ideally, the inventive methods prophylactically or therapeutically treat an animal, preferably a mammal (e.g., a human), for at least one disorder associated with loss or damage of sensory hair cells, e.g., disorders of the ear associated with damage of sensory hair cells (such as hearing loss or balance disorders). Inventive methods also are useful in maintaining a level of sensory perception, i.e., controlling the loss of perception of environmental stimuli caused by, for instance, the aging process or ototoxic agents. Inhibitors of EGFR and proteins downstream or associated therewith provide a therapeutic effect.

An inhibitor of this invention can selectively decrease or block the expression of an EGFR signaling protein (i.e., transcription or translation of the protein), decrease or block the activity of an EGFR signaling protein (i.e., binding to ligands, tyrosine kinase activity, phosphorylation, protein-protein interactions, and/or downstream signaling), decrease or block the biological effect(s) of an EGFR signaling protein, and/or modify half-life or subcellular localization (membrane versus cytoplasmic or nuclear localization, internalization, and recycling) of an EGFR signaling protein. In particular, an inhibitor of EGFR signaling is an active agent that selectively decreases or blocks one or more of the following: transcription or translation, ligand binding, phosphorylation, multimerization, tyrosine kinase activity, internalization, and/or translocation into the nucleus.

Ideally, EGFR signaling is completely blocked, or is reduced by at least 10%, at least 20%, at least 30%, at least 40%, at least 50%, at least 60%, at least 70%, at least 75%, at least 80%, at least 85%, at least 90%, at least 92.5%, at least 95%, at least 97%, at least 98%, at least 98.5%, at least 99%, at least 99.25%, at least 99.5%, or at least 99.75% by the inhibitor of EGFR signaling inhibitor as compared to normal physiologic levels.

The inhibitor of EGFR signaling of this invention typically has a half maximal (50%) inhibitory concentration (IC₅₀) in the range of 1 μM to 100 μM. Preferably, the inhibitor of EGFR signaling has an IC₅₀ value of less than 10 μM, less than 5 μM, less than 1 μM, or less than 100 nM. Moreover, in some embodiments, the inhibitor of EGFR signaling is specific/selective for one or more of the EGFR signaling proteins of interest and fails to inhibit or inhibit to a substantially lesser degree other non-EGFR pathway proteins. In this respect, it is preferable that the inhibitor of EGFR signaling is a selective inhibitor of EGFR signaling. Preferably, selectivity is for one, two, three or four EGFR signaling proteins and fails to inhibit or inhibits to a substantially lesser degree other non-EGFR pathway proteins. By way of illustration, an inhibitor can be a dual EGFR and ERBB2 inhibitor, both of which are EGFR signaling proteins. Methods for assessing the selectively of inhibitors are known in the art and can be based upon any conventional assay including, but not limited to the determination of the IC₅₀, the binding affinity of the inhibitor (i.e., Kᵢ), and/or the half maximal effective concentration (EC₅₀) of the inhibitor for EGFR signaling protein of interest as compared to another protein (comparative protein). In particular embodiments, a selective inhibitor of EGFR signaling is an inhibitor that has an IC₅₀ value for an EGFR signaling protein of interest that is at least twice or, more desirably, at least three, four, five, or six times lower than the corresponding IC₅₀ value for a comparative protein. Most desirably, a selective inhibitor of EGFR signaling has an IC₅₀ value for an EGFR signaling protein which is at least one order of magnitude or at least two orders of magnitude lower than the IC₅₀ value for a comparative protein.

Sensory Perception. The invention provides for the modulation of sensory perception in an animal by administering to the inner ear an inhibitor of EGFR signaling and optionally an expression vector (e.g., expression viral vector) harboring a nucleic acid molecule encoding an otoprotective agent. By “modulating sensory perception” it means achieving, at least in part, the ability to recognize and adapt to environmental changes. In terms of sensory hair cell function, modulation in sensory perception is associated with the generation or protection of sensory hair cells that convert mechanical stimuli in the inner ear into neural impulses, which are then processed in the brain such that an animal is aware of environmental change, e.g., sound, language, or body/head position. Sensory hair cells are preferably generated in the organ of Corti and/or vestibular apparatus. In the context of prophylaxis, sensory hair cells, which would otherwise be initially or further damaged or lost due to, e.g., ototoxic agents, are protected from damage or loss by the administration of an inhibitor of EGFR signaling and optionally an otoprotective agent.

A change in the ability of a subject to detect sound is readily accomplished through administration of simple hearing tests, such as a tone test commonly administered by an audiologist. In most mammals, a reaction to different frequencies indicates a change in sensory perception. In humans, comprehension of language also is appropriate. For example, it is possible for a subject to here while being unable to understand speech. A change in perception is indicated by the ability to distinguish different types of acoustic stimuli, such as differentiating language from background noise, and by understanding speech. Speech threshold and discrimination tests are useful for such evaluations.

Evaluation of changes in balance, motion awareness, and/or timing of response to motion stimuli also is achieved using a variety of techniques. Vestibular function also can be measured by comparing the magnitude of response to motion stimulus (gain) or timing of initiation of response (phase). Animals can be tested for Vestibulo-Ocular Reflex (VOR) gain and phase using scleral search coils to evaluate improvements in sensory perception. Electronystagmography (ENG) records eye movements in response to stimuli such as, for instance, moving or flashing lights, body repositioning, fluid movement inside the semicircular canals, and the like. Evaluation of balance during movement using a rotating chair or moving platform is also useful in this respect.

To detect a change in sensory perception, a baseline value is recorded prior to the inventive method using any appropriate sensory test. A subject is reevaluated at an appropriate time period following the inventive method (e.g., 1 hour, 6 hours, 12 hours, 18 hours, 1 day, 3 days, 5 days, 7 days, 14 days, 21 days, 28 days, 2 months, 3 months or more following the inventive method), the results of which are compared to baseline results to determine a change in sensory perception.

Method of Prevention or Treatment. The inventive method promotes the protection and/or generation of sensory hair cells that allow perception of stimuli. Accordingly, this invention provides a method for the prevention, treatment, control, amelioration, or reduction of risk of hearing impairments, loss, and disorders by administering to a subject in need of treatment an inhibitor of EGFR signaling and/or one or more otoprotective/regenerative agents. Ideally, the inventive method prophylactically or therapeutically treats an animal for at least one disorder associated with loss, damage, absence of sensory hair cells, such as hearing loss and balance disorders. Hearing loss can be caused by damage of hair cells of the organ of Corti due to bacterial or viral infection, heredity, physical injury, acoustic trauma, ototoxic drugs (e.g., aminoglycoside antibiotic or cisplatin) and the like. While hearing loss is easily identified, balance disorders manifest in a broad variety of complications easily attributable to other ailments. Symptoms of a balance disorder include disorientation, dizziness, vertigo, nausea, blurred vision, clumsiness, and frequent falls. Balance disorders treated by the inventive method preferably involve a peripheral vestibular disorder (i.e., a disturbance in the vestibular apparatus) involving dysfunctional translation of mechanical stimuli into neural impulses due to damage or lack of sensory hair cells.

In one aspect, methods of protecting against or preventing hearing loss or impairment are provided. In accordance with such methods, a subject in need of treatment is administered an effective amount of inhibitor of EGFR signaling. In some embodiments, the inhibitor of EGFR signaling inhibits the expression or activity of EGFR, a Ras/Raf/MEK/ERK/MAPK protein, a JAK/STAT protein, a PI3K/AKT/mTOR protein, an NCK-PAK-JNK protein, a PLC-DAG-PKC protein, or a cell cycle-associated protein kinase associated with or downstream of EGFR. In other embodiments, prevention of hearing loss is achieved by administering to a subject in need of treatment an inhibitor of a cell cycle-associated protein kinase associated with or downstream of EGFR. In certain embodiments, prevention of hearing loss is achieved by administering to a subject in need of treatment an inhibitor of Her-2, Aurora kinase, B-Raf, or PDGFR expression or activity. The inhibitor of EGFR signaling can be administered alone or in combination with one or more otoprotective agents. The term “otoprotective agent” refers to an agent that reduces or prevents noise-induced hearing loss, chemically induced hearing loss, or age-induced hearing impairment or otherwise protects against hearing impairment. Examples of otoprotective agents include, but are not limited to, PARP-1 inhibitors; pirenzepine LS-75, otenzepad, AQ-RA741, viramune, BIBN 99, DIBD, telenzepine (see US 2011/0263574); methionine (see U.S. Pat. No. 7,071,230); IGF-1, FGF-2, aspirin, reduced glutathione, N-methyl-(D)-glucaminedithiocarbamate, and iron chelators such as tartrate and maleate. See also US 2005/0101534 for additional otoprotective agents.

Protection against and prevention or treatment of hearing loss or impairment can be in the context of conditions including, but not limited to, tinnitus, ringing, Presbyacusis, auditory neuropathy, acoustic trauma, acoustic neuroma, Pendred syndrome, Usher syndrome, Wardenburg syndrome, non-syndromic sensorineural deafness, otitis media, otosclerosis, Meniere's disease, ototoxicity, labyrinthitis, as well as hearing impairments caused by infection (i.e., measles, mumps, or meningitis), medicines such as antibiotics, and some cancer treatments (i.e., chemotherapy and radiation therapy).

In certain embodiments, the hearing impairment is drug-induced. In a still further aspect, the drug is a chemotherapeutic agent. More specifically, the drug is a platinum-based chemotherapeutic agent such as carboplatin, cisplatin, transplatin, nedaplatin, oxaliplatin, picoplatin, satraplatin, transplatin, and triplatin, or a pharmaceutically acceptable salt thereof. In a particular embodiment, the platinum-based chemotherapeutic agent is cisplatin, or a pharmaceutically acceptable salt thereof. In yet a further embodiment, the drug is an antibiotic, including, but not limited to, daunorubicin, doxorubicin, epirubicin, idarubicin, actinomycin-D, bleomycin, mitomycin-C, amikacin, apramycin, arbekacin, astromicin, bekanamycin, dibekacin, framycetin, gentamicin, hygromycin B, isepamicin, kanamycin, neomycin, netilmicin, paromomycin, rhodostreptomycin, ribostamycin, sisomicin, spectinomycin, streptomycin, tobramycin, and verdamicin, or a pharmaceutically acceptable salt thereof.

In a further aspect, the hearing impairment is age-related, noise-induced or a balance or orientation-related disorder. Examples of balance disorders include, but are not limited to, induced or spontaneous vertigo, dysequilibrium, increased susceptibility to motion sickness, nausea, vomiting, ataxia, labyrinthitis, oscillopsia, nystagmus, syncope, lightheadedness, dizziness, increased falling, difficulty walking at night, Meniere's disease, and difficulty in visual tracking and processing. Further, the noise-induced hearing loss may be temporary or permanent.

More than one billion teens and young adults worldwide are at risk of hearing loss from exposure to loud music, as recently reported by the World Health Organization. Many other noise exposures, including occupational settings and consumer-operated devices, also cause noise-induced hearing loss, which is among the most common physical complaints, and which detracts significantly from the ability to converse, communicate, and participate in everyday life (thus reducing general quality of life of the individual and the family). Acute or chronic acoustic overexposure has put more than 40 million US workers at risk of permanent hearing loss (Kopke, et al. (2007) Hear. Res. 226:114-125).

Traumatic brain injury (TBI) and blast-associated injury occur most frequently in military situations where blast exposure cannot be predicted, trauma intensity exceeds the effectiveness of protective devices, or protective devices are not available. TBI is often accompanied by a diverse range of disruptions or damage to the auditory sensory system, which is highly vulnerable to blast injury. Extreme physical blast force can cause damage of various types to the peripheral auditory system, including rupture of the tympanic membrane (TM, eardrum), fracture of the middle ear bones, dislocation of sensory hair cells from the basilar membrane, and loss of spiral ganglia that innervate hair cells. In human studies of blast injury, approximately 17-29% of cases involve severe TM rupture, while 33-78% involve moderate to severe sensorineural hearing loss (hair cell and ganglion loss). Therefore, TBI and blast injury are a common, although extreme, cause of hearing loss.

Biological protection of hearing is more promising than currently available mechanical protective devices. Hearing aids are frequently problematic because of their high cost and their many technical issues. Ideally, service men and women could take protective drugs before entering high-risk or high-noise settings and would then be protected from noise injury with no effect on performance. To date, there are no FDA-approved drugs for protection against noise- and TBI-associated hearing loss.

In accordance with the methods of this invention, the inhibitor of EGFR signaling can be administered locally, e.g., to the inner ear of the subject. Alternatively, the inhibitor of EGFR signaling can be administered systemically. Further, the inhibitor of EGFR signaling can be administered via injection into one or more of the scala tympani, cochlear duct, scala vestibule of the cochlea, into the auditory nerve trunk in the internal auditory meatus, or into the middle ear space across the transtympanic membrane/ear drum. Moreover, when used in combination, the EGFR signaling can be administered via the same or different routes.

In various aspects, the disclosed molecules can be used in combination with one or more other drugs in the treatment, prevention, control, amelioration, or reduction of risk of hearing impairments and disorders for which disclosed molecules, or the other drugs can have utility, where the combination of the drugs together are safer or more effective than either drug alone. Such other drug(s) can be administered, by a route and in an amount commonly used therefore, contemporaneously or sequentially with a compound of the present invention. When a molecule of the present invention is used contemporaneously with one or more other drugs, a pharmaceutical composition in unit dosage form containing such other drugs and a disclosed compound is preferred. However, the combination therapy can also include therapies in which a disclosed molecule and one or more other drugs are administered on different overlapping schedules. It is also contemplated that when used in combination with one or more other active ingredients, the disclosed molecules and the other active ingredients can be used in lower doses than when each is used singly.

The methods herein are useful in the prevention or treatment of both acute and persistent, progressive disorders associated with lack of or damage to functional sensory hair cells. For acute ailments, the drugs herein can be administered using a single application or multiple applications within a short time period. For persistent diseases, such as hearing loss, or disorders stemming from a massive loss of sensory hair cells, numerous rounds of administration of the drugs herein may be necessary to realize a therapeutic effect.

Where appropriate, following treatment, the subject (e.g., human or other animal) can be tested for an improvement in hearing or in other symptoms related to hearing disorders. Subjects benefiting from treatment include those at risk of hair cell loss. For example, a subject having or at risk for developing a hearing loss can hear less well than the average subject (e.g., an average human being), or less well than a subject before experiencing the hearing loss. For example, hearing can be diminished by at least 5%, 10%, 30%, 50% or more. Methods for measuring hearing are well-known and include pure tone audiometry, air conduction, and bone conduction tests. These exams measure the limits of loudness (intensity) and pitch (frequency) that a human can hear. Hearing tests in humans include behavioral observation audiometry (for infants to seven months), visual reinforcement orientation audiometry (for children 7 months to 3 years) and play audiometry for children older than 3 years. Oto-acoustic emission testing can be used to test the functioning of the cochlear hair cells, and electro-cochleography provides information about the functioning of the cochlea and the first part of the nerve pathway to the brain. In various aspects, treatment can be continued with or without modification or can be stopped.

Routes of Administration. One skilled in the art will appreciate that suitable methods of administering a drug to the inner ear are available. Although more than one route can be used to administer a particular drug or expression vector, a particular route can provide a more immediate and more effective reaction than another route. Accordingly, the described routes of administration are merely exemplary and are in no way limiting.

No matter the route of administration, a drug or expression vector of the inventive method ideally reaches the sensory epithelium of the inner ear. The most direct routes of administration, therefore, entail surgical procedures which allow access to the interior of the structures of the inner ear. Inoculation via cochleostomy allows administration of an expression vector directly to the regions of the inner ear associated with hearing. Cochleostomy involves drilling a hole through the cochlear wall, e.g., in the otic capsule below the stapedial artery as described in Kawamoto, et al. ((2001) Molecular Therapy 4 (6): 575-585), and release of a pharmaceutical composition containing the drug or expression vector. Administration to the endolymphatic compartment is particularly useful for administering an adenoviral vector to the areas of the inner ear responsible for hearing. Alternatively, a drug or expression vector can be administered to the semicircular canals via canalostomy. Canalostomy provides for transgene expression in the vestibular system and the cochlea, whereas cochleostomy does not provide as efficient transduction in the vestibular space. The risk of damage to cochlear function is reduced using canalostomy in as much as direct injection into the cochlear space can result in mechanical damage to hair cells (Kawamoto, et al., supra). Administration procedures also can be performed under fluid (e.g., artificial perilymph), which can include factors to alleviate side effects of treatment or the administration procedure, such as apoptosis inhibitors or anti-inflammatoires. Another direct route of administration to the inner ear is through the round window, either by injection or topical application to the round window.

A drug can be present in a pharmaceutical composition for administration to the inner ear. In certain cases, it may be appropriate to administer multiple applications and/or employ multiple routes, e.g., canalostomy and cochleostomy, to ensure sufficient exposure of supporting cells to the drug or expression vector.

A drug or expression vector can be present in or on a device that allows controlled or sustained release of the drug or expression vector, such as a sponge, meshwork, mechanical reservoir or pump, or mechanical implant. For example, a biocompatible sponge or gelform soaked in a pharmaceutical composition containing the drug or expression vector is placed adjacent to the round window, through which the drug or expression vector permeates to reach the cochlea (as described in Jero, et al., supra). Mini-osmotic pumps provide sustained release of a drug or expression vector over extended periods of time (e.g., five to seven days), allowing small volumes of composition containing the drug or expression vector to be administered, which can prevent mechanical damage to endogenous sensory cells. The drug or expression vector also can be administered in the form of sustained-release formulations (see, e.g., U.S. Pat. No. 5,378,475) containing, for example, gelatin, chondroitin sulfate, a polyphosphoester, such as bis-2-hydroxyethyl-terephthalate (BHET), or a polylactic-glycolic acid.

Alternatively, the drug or expression vector can be administered parenterally, intramuscularly, intravenously, orally or intraperitoneally. As discussed herein, an expression vector can be modified to alter the binding specificity or recognition of an expression vector for a receptor on a potential host cell. With respect to adenovirus, such manipulations can include deletion of regions of the fiber, penton, or hexon, insertions of various native or non-native ligands into portions of the coat protein, and the like. One of ordinary skills in the art will appreciate that parenteral administration can require large doses or multiple administrations to effectively deliver the expression vector to the appropriate host cells. Pharmaceutically acceptable carriers for compositions are well-known to those of ordinary skill in the art (see Pharmaceutics and Pharmacy Practice, (1982) J. B. Lippincott Co., Philadelphia, PA, Banker and Chalmers, eds., pages 238-250; ASHP Handbook on Injectable Drugs (1986) Toissel, 4^{th} ed., pages 622-630). Although less preferred, the expression vector can also be administered in vivo by particle bombardment, i.e., a gene gun.

One of ordinary skills in the art also will appreciate that dosage and routes of administration can be selected to minimize loss of expression vector due to a host's immune system. For example, for contacting target cells in vivo, it can be advantageous to administer to a host a null expression vector (i.e., an expression vector not harboring the nucleic acid molecule(s) of interest) prior to performing the inventive method. Prior administration of null expression vectors can serve to create immunity in the host to the expression vector hinders the body's innate clearance mechanisms, thereby decreasing the amount of vector cleared by the immune system.

Dosage. The dose of a drug or expression vector administered to an animal, particularly a human, in accordance with the invention should be sufficient to affect the desired response in the animal over a reasonable time frame. One skilled in the art will recognize that dosage will depend upon a variety of factors, including the age, species, location of damaged sensory epithelia, the pathology in question (if any), and condition or disease state. Dosage also depends on the inhibitor of EGFR signaling and/or cell cycle-associated protein kinase inhibitor, as well as the amount of sensory epithelium to be transduced. The size of the dose also will be determined by the route, timing, and frequency of administration as well as the existence, nature, and extent of any adverse side effects that might accompany the administration of a particular expression vector (e.g., surgical trauma) or drug and the desired physiological effect. It will be appreciated by one of ordinary skill in the art that various conditions or disease states, in particular, chronic conditions or disease states, may require prolonged treatment involving multiple administrations. Suitable doses and dosage regimens can be determined by conventional range-finding techniques known to those of ordinary skill in the art. One skilled in the art can extrapolate the dose conversion between animals and humans. See e.g. A. Nair, A Simple Practice Guide for Dose Conversion Between Animals and Humans. J. of Basic and Clin. Pharmacy, V. 7, Issue 2 March-May (2017).

The interior space of the structures of the inner ear is limited. The volume of pharmaceutical composition administered directly into the inner ear structures should be carefully monitored, as forcing too much composition will damage the sensory epithelium. For a human patient, the volume administered is preferably about 10 μl to about 2 ml (e.g., from about 25 μl to about 1.5 ml) of composition. For example, from about 50 μl to about 1 ml (e.g., about 100 μl, 200 μl, 300 μl, 400 μl, 500 μl, 600 μl, 700 μl, 800 μl or 900 μl) of composition can be administered. In one embodiment, the entire fluid contents of the inner ear structure, e.g., the cochlea or semi-circular canals, is replaced with pharmaceutical composition. In another embodiment, a pharmaceutical composition of the invention is slowly released into the inner ear structure, such that mechanical trauma is minimized.

It can be advantageous to administer two or more (i.e., multiple) doses of the drug which is an inhibitor of EGFR signaling. The inventive method provides for administration of multiple doses of a drug or expression vector to change the sensory perception of an animal. For example, at least two doses of a drug or expression vector can be administered to the same ear. Preferably, multiple doses are administered while retaining gene expression above background levels. Also preferably, the sensory epithelium of the inner ear is contacted with two doses or more of the drug or expression vector within about 30 days. Preferably, two or more applications are administered to the inner ear within about 90 days. However, three, four, five, six, or more doses can be administered in any time frame (e.g., 2, 7, 10, 14, 21, 28, 35, 42, 49, 56, 63, 70, 77, 85 or more days between doses).

Pharmaceutical Composition. A drug or expression vector of the invention desirably is administered in a pharmaceutical composition, which includes a pharmaceutically acceptable carrier and the drug or expression vector(s). Any suitable pharmaceutically acceptable carrier can be used within the context of the invention, and such carriers are well known in the art. The choice of carrier will be determined, in part, by the particular site to which the composition is to be administered and the particular method used to administer the composition. Ideally, in the context of adenoviral vectors, the pharmaceutical composition preferably is free of replication-competent adenovirus.

Suitable formulations include aqueous and non-aqueous solutions, isotonic sterile solutions, which can contain antioxidants, buffers, bacteriostats, and solutes that render the formulation isotonic with the blood or fluid of the inner ear of the intended recipient, and aqueous and non-aqueous sterile suspensions that can include suspending agents, solubilizers, thickening agents, stabilizers, and preservatives. The formulation can include artificial endolymph or perilymph, which are commercially available. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Preferably, the pharmaceutically acceptable carrier is a buffered saline solution. More preferably, the expression vector for use in the inventive method is administered in a pharmaceutical composition formulated to protect the expression vector from damage prior to administration. For example, pharmaceutical composition can be formulated to reduce loss of the expression vector on devices used to prepare, store, or administer the expression vector, such as glassware, syringes, or needles. The pharmaceutical composition can be formulated to decrease the light sensitivity and/or temperature sensitivity of the expression vector. To this end, the pharmaceutical composition preferably includes a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a pharmaceutical composition will extend the shelf-life of the vector, facilitate administration, and increase the efficiency of the inventive method. In this regard, a pharmaceutical composition can also be formulated to enhance transduction efficiency. In addition, one of the ordinary skills in the art will appreciate that the expression vector, e.g., viral vector, can be present in a composition with other therapeutic or biologically active agents. For example, therapeutic factors useful in the treatment of a particular indication can be present. Factors that control inflammation, such as ibuprofen or steroids, can be part of the composition to reduce swelling and inflammation associated with in vivo administration of the viral vector. Immune system suppressors can be administered in combination with the inventive method to reduce any immune response to the vector itself or associated with a disorder of the inner ear. Angiogenic factors, neurotrophic factors, proliferating agents, and the like can be present in pharmaceutical composition. Similarly, vitamins and minerals, antioxidants, and micronutrients can be co-administered. Antibiotics, i.e., microbicides and fungicides, can be present to reduce the risk of infection associated with gene transfer procedures and other disorders.

As discussed herein, several options are available for delivering multiple coding sequences to the inner ear. The nucleic acid molecule(s) encoding the inhibitor of EGFR signaling can encode additional gene products. The expression vector alternatively, or in addition, can include multiple expression cassettes encoding different gene products. Multiple coding sequences can be operably linked to different promoters, e.g., different promoters having dissimilar levels and patterns of activity. Alternatively, the multiple coding sequences can be operably linked to the same promoter to form a polycistronic element. The invention also contemplates administering to the inner ear a cocktail of expression vectors, wherein each expression vector encodes a gene product beneficial to sensory perception. The cocktail of expression vectors can further comprise different types of expression vectors, e.g., adenoviral vectors and adeno-associated viral vectors.

The method of the invention can be part of a treatment regimen involving other therapeutic modalities. It is appropriate, therefore, if the inventive method is employed to prophylactically or therapeutically treat a sensory disorder, namely a hearing disorder or a balance disorder, that has been treated, is being treated, or will be treated with any of a number of other therapies, such as drug therapy or surgery. The inventive method also can be performed in conjunction with the implantation of hearing devices, such as cochlear implants. The inventive method also is particularly suited for procedures involving stem cells to regenerate populations of cells within the inner ear. In this respect, the inventive method can be practiced ex vivo to transduce stem cells, which are then implanted within the inner ear.

The inventive method also can involve the co-administration of other pharmaceutically active compounds. By “co-administration” means administration before, concurrently with, e.g., in combination with the expression vector in the same formulation or in separate formulations, or after administration of the expression vector as described above. For example, factors that control inflammation, such as ibuprofen or steroids, can be co-administered to reduce swelling and inflammation associated with administration of the expression vector. Immunosuppressive agents can be co-administered to reduce inappropriate immune responses related to an inner ear disorder or the practice of the inventive method. Similarly, vitamins and minerals, antioxidants, and micronutrients can be co-administered. Antibiotics, i.e., microbicides and fungicides, can be co-administered to reduce the risk of infection associated with surgical procedures.

The following non-limiting examples are provided to further illustrate the present invention.

### Materials and Methods

### Mouse Model

For the single-dose cisplatin protocol, FVB/NJ breeding mice were purchased from Jackson Laboratory (Bar Harbor, Maine, USA), bred in the animal facility at Creighton University, and used at 6-8 weeks old for the single dose cisplatin experiment. For the multi-cycle cisplatin protocol, 8-week-old CBA/CaJ mice were purchased from Jackson Laboratory with an equal number of males and females. The CBA/CaJ mice were given one week to acclimate to the Animal Resource Facilities at Creighton University. Animals were anesthetized by Avertin (2,2,2-tribromoethanol) via intraperitoneal injection at a dose of 500 mg/kg, and complete anesthetization was determined via toe pinch. For all experiments, mice were randomly assigned to experimental groups, maintaining a balance of males and females in each group.

### Single Dose Cisplatin Treatment in Mice

10 milligrams of cisplatin (479306, Sigma-Aldrich) powder were dissolved in 10 mL of sterile saline (0.9% NaCl) at 37° C. for 40 to 60 minutes. 30 mg/kg was administered once to FVB mice via intraperitoneal injection on day 1 of the protocol (FIG. 1A) (6, 20). One day before cisplatin injection, mice received 1 mL of saline by subcutaneous injection and were given 1 mL of saline twice a day throughout the protocol until body weight started to recover. The cages of cisplatin treated mice were placed on heating pads until body weights began to recover. Food pellets dipped in DIETGEL Boost (ClearH20 Westbrook, Maine were placed on the cage floor of cisplatin-treated mice. DIETGEL Boost (ClearH20 Westbrook, Maine is a high calorie dietary supplement that provides extra calorie support for mice. The investigators and veterinary staff carefully monitored for changes in overall health and activity that may have resulted from cisplatin treatment.

### Multi-Cycle Cisplatin Treatment in Mice

4.5 milligrams of cisplatin (479306, Sigma-Aldrich) powder were dissolved in 25 mL of sterile saline (0.9% NaCl) at 37° C. for 40 to 60 minutes. 3 mg/kg cisplatin was administered to mice via intraperitoneal injection once a day in the morning. This repeated for 4 total days with a 10-day recover period in which no cisplatin was administered to the mice. Mice were treated with 3 mg/kg cisplatin for a total of 12 days (4 days per cycle with 3 cycles) (FIG. 2A) (46,47). Cisplatin treated mice were injected by subcutaneous injection twice a day with 1 mL of warm saline to ameliorate dehydration. This continued until my body weight started to recover. The cages of cisplatin-treated mice were placed on heating pads throughout the duration of the experiment until mice began to recover after the 3rd treatment cycle of the protocol. Food pellets dipped in DIETGEL Boost (ClearH20 Westbrook, Maine). were placed on the cage floor of cisplatin-treated mice. The investigators and veterinary staff carefully monitored for changes in overall health and activity that may have resulted from cisplatin treatment.

Compound administration by oral gavage. The compound dabrafenib mesylate was purchased from MedChemExpress and administered to FVB/NJ and CBA/CaJ mice via oral gavage. Dabrafenib was dissolved in a mixture of 10% DMSO, 5% Tween 80, 40% PEG-E-300, and 45% saline. For the single dose cisplatin experiment, 12 mg/kg dabrafenib was given to mice once in the morning and once at night. This continued for a total of 3 days (FIG. 1A). For the multi-cycle cisplatin protocol, 15, 3, or 0.6 mg/kg dabrafenib was administered once in the morning and once at night for 4 total days with a 10-day recovery period in which no dabrafenib was administered to the mice. This cycle was repeated a total of 3 times (FIG. 2A). Mice treated with cisplatin and dabrafenib were given dabrafenib 1 hour before treatment with cisplatin in the morning.

### ABR Threshold and Wave 1 Amplitude Measurements

ABR waveforms in anesthetized mice were recorded in a sound booth by using subdermal needles positioned in the skull, below the pinna, and at the base of the tail, and the responses were fed into a low-impedance Medusa digital biological amplifier system (RA4L; TDT; 20-dB gain). At the tested frequencies (8, 16, and 32 kHz), the stimulus intensity was reduced in 10-dB steps from 90 to 10 dB to determine the hearing threshold. ABR waveforms were averaged in response to 500 tone bursts with the recorded signals filtered by a band-pass filter from 300 Hz to 3 kHz. ABR threshold was determined by the presence of at least 3 of the 5 waveform peaks (6, 20). Baseline ABR recordings before any treatment were performed when mice were 6-7 weeks old for the single dose cisplatin experiments and 9 weeks old for the multi-dose cisplatin protocol. All beginning threshold values were between 10 and 40 dB at all tested frequencies. In the single dose cisplatin experiment, post-treatment recordings were performed 21 days following cisplatin treatment. For the multi-cycle cisplatin protocol, post-treatment recordings were performed 42 days after the start of the 3-cycle protocol (aged 18 weeks) with half the mice kept alive and ABR was performed again on these mice 4 months after the completion of the 42-day treatment protocol. All thresholds were determined independently by two-three experimenters for each mouse who were blind to the treatment the mice received. ABR wave one amplitudes were measured as the difference between the peak of wave 1 and the noise floor of the ABR trace.

### DPOAE Measurements

Distortion product otoacoustic emissions were recorded in a sound booth while mice were anesthetized. DPOAE measurements were recorded using the TDT RZ6 processor and BioSigTZ software. The ER10B+ microphone system was inserted into the ear canal in a way that allowed for the path to the tympanic membrane to be unobstructed. DPOAE measurements occurred at 8, 16, and 32 KHz with an f2/f1 ratio of 1.2. Tone 1 was *.909 of the center frequency and tone 2 was *1.09 of the center frequency. DPOAE data was recorded every 20.97 milliseconds and average 513 times at each intensity level and frequency. At each tested frequency, the stimulus intensity was reduced in 10 dB steps starting at 90 dB and ending at 10 dB. DPOAE threshold was determined by the presence of an emission above the noise floor. Baseline DPOAE recordings occurred when CBA/CaJ mice were 10 weeks old and tested again on day 42 (immediately after cycle 3) and on day 165 (4 months after cycle 3). DPOAE threshold shifts were determined by subtracting the baseline DPOAE recording from the post experimental recording.

### Tissue Preparation, Immunofluorescence, and OHCs Counts.

Cochleae from adult mice were prepared and examined as described previously (80-82). Cochleae samples were immunostained with anti-myosin VI (1:400; 25-6791, Proteus Bioscience) or pERK antibody (1:400; 9101 L, Cell Signaling) with secondary antibodies purchased from Invitrogen coupled to anti-rabbit Alexa Fluor 488 (1:400; A11034). All images were acquired with a confocal microscope (LSM 700 or 710, Zeiss). Outer hair cell counts were determined by the total amount of outer hair cells in a 160 μm region (6, 20, 82). Counts were determined for the 8, 16, and 32 kHz regions. Cochleae from each experimental group were randomly selected to be imaged for outer hair cell counts.

### Endocochlear Potential Measurements

Mice were anesthetized using a combined regimen of ketamine (16.6 mg/ml) and xylazine (2.3

mg/ml) and supplemented as needed to maintain a surgical level via intraperitoneal injection. For recording the EP, a round-window approach was used. A glass capillary pipette electrode (10 MU) was mounted on a hydraulic micromanipulator and advanced until a stable positive potential was observed. Signals were filtered and amplified under current-clamp mode using an Axopatch 200B amplifier (Molecular Devices, San Jose, CA) and acquired by software pClamp 9.2. The sampling frequency was 10 KHz.

Kidney histology examination. Following cisplatin and dabrafenib treatment, mice were sacrificed, and kidneys were extracted and put into 4% PFA. The kidneys were later embedded in paraffin, sectioned (3 μm), and stained with Hematoxylin and Eosin (H&E) and Periodic Acid-Schiff (PAS). Sections were observed under a microscope (Nikon Eclipse Ci) for histological examination. A semi-quantitative pathological scoring system was used as described in Pabla et al, 2015 and Hu et al, 2010 (54, 55). The grading system uses scores of 0-4 that indicate the percentage of damage in each section. Sections were analyzed by an experienced pathologist in a double-blind manner. The grades are: grade 0 (minimal)=<10% damage with no visible lesions and normal morphology; grade 1 (mild)=11-25% damage with mild tubule dilation, swelling of cells, presence of luminal debris or cast and nuclear condensation with partial loss of brush borders in 1/3 tubules; grade 2 (moderate)=26-50% damage with clear dilation of tubules, loss of brush borders, nuclear loss and presence of casts in <2/3 of tubules; Grade 3 (marked)=51-75% damage with severe dilation of most tubule, total loss of brush borders and nuclear loss in 2/3 tubule and grade 4 (severe)=>75% damage with complete loss of tissue morphology, severe tubule dilation and loss of nucleus and brush borders.

Liver histology examination. Following cisplatin and dabrafenib treatment, mice were sacrificed, and livers were extracted and put into 4% PFA. The livers were later embedded in paraffin, sectioned (3 μm), and stained with Hematoxylin and Eosin (H&E) and Masson's trichrome stain. Sections were observed under a microscope (Nikon Eclipse Ci) for histological examination. The grading system uses a score of 0-4 that indicates the amount of damage in each section. Sections were analyzed by an experienced pathologist in a double-blind manner. The grades are grade 0 (normal), grade 1 (mild damage), grade 2 (moderate damage), grade 3 (severe damage), and grade 4 (very severe/fulminant damage). Criteria that determined the scoring of each liver sample was the presence of fibrosis, lobular disarray, hepatocyte swelling, hepatocyte nuclear changes, hepatocyte necrosis, lobular inflammation, portal inflammation, sinusoidal and central vein congestion, and Kupffer cell hyperplasia.

Statistical Analysis. Statistics was performed using Prism (GraphPad Software). A two-way analysis of variance (ANOVA) with Bonferroni post hoc test was used to determine mean difference and statistical significance. Statistical significance was determined when P<0.05.

The following non-limiting examples are provided to further illustrate the present invention.

### Example 1: EGFR Inhibitors for Protection Against Hearing Loss

A screen of a library composed of 75 kinase inhibitors was conducted to identify inhibitors that protect against cisplatin-induced hair cell loss. This screen identified four compounds: (1) Her2 inhibitor MUBRITINIB (TAK 165), (2) Pan-AUR inhibitor SNS314 (3) BRAF-V600E inhibitor GSK2118436A (DABRAFENIB), and (4) PDGFR inhibitor CRENOLANIB that potently protected against cisplatin-induced cell death in a mouse cochlea-derived cell line (HEI-OC1) as well as cisplatin-induced hair cell loss in mouse cochlear explants.

Her2 inhibitor MUBRITINIB (TAK 165) exhibited protective effects against cisplatin-induced Caspase-3/7 activity in HEI-OC1 cells with an IC₅₀ of 4 nM and LD₅₀ of >55 μM; and protected against cisplatin-induced hair cell loss in mouse cochlear explants with IC₅₀ of 2.5 nM and LD₅₀ of >500 nM (Therapeutic Index of >200) (FIG. 1). Similarly, the pan-ErbB inhibitor, PELITINIB, was found to exhibit protective effects against cisplatin-induced Caspase-3/7 activity in HEI-OC1 cell loss with IC₅₀ of 0.6 μM and LD₅₀ of 40 μM (FIG. 2). Moreover, with 1 hour pre-incubation, PELITINIB exhibited 49% protection of outer hair cells against cisplatin-induced hair cell loss in mouse cochlear explants (N=3).

Similarly, the pan-ErbB inhibitor, PELITINIB, was found to exhibit protective effects against cisplatin-induced Caspase-3/7 activity in HEI-OC1 cell loss with IC₅₀ of 0.6 μM and LD₅₀ of 40 μM (FIG. 2). Moreover, with 1 hour pre-incubation, PELITINIB exhibited 49% protection of outer hair cells against cisplatin-induced hair cell loss in mouse cochlear explants (N=3).

B-Raf inhibitors protected outer hair cells against cisplatin injury in mouse cochlear explants. B-Raf inhibitor Dabrafenib (BRAF) (FIG. 3A) exhibited protective cisplatin-induced hair cell loss in mouse cochlear explants with IC₅₀ of 0.0300 μM and LD₅₀ of 13.47 μM (Therapeutic Index of greater than 2000). Additionally, Dabrafenib (BRAF) showed Zebrafish compound cisplatin protection at 0.100 μM (FIG. 11A).

Additionally, the B-Raf inhibitor VEMURAFENIB (BRAF) (FIG. 4B) exhibited protective cisplatin-induced hair cell loss in mouse cochlear explants with IC₅₀ of ˜0.2 μM and LD₅₀ of greater than 3 μM (Therapeutic Index of greater than 15). B-Raf inhibitor PLX-4750 (BRAF) (FIG. 4D) exhibited protective cisplatin-induced hair cell loss in mouse cochlear explants with IC₅₀ of 0.2 μM and LD₅₀ of greater than 3 μM. (Therapeutic Index of greater than 15). B-Raf inhibitor RAF-265 (BRAF) (FIG. 4E) exhibited protective cisplatin-induced hair cell loss in mouse cochlear explants with IC₅₀ of 0.02 μM and LD₅₀ of greater than 3 μM (Therapeutic Index of greater than 150).

MEK inhibitors protected outer hair cells against cisplatin injury in cochlear explants as shown in FIG. 4C MEK inhibitor TRAMETINIB (MEK) exhibited protective cisplatin-induced hair cell loss in mouse cochlear explants with IC₅₀ of 0.05 μM and LD₅₀ of greater than 3 μM (Therapeutic Index of greater than 60).

As illustrated in FIG. 4A inhibitor mitigates cisplatin along the signaling cascade.

### Example 2 Inhibitors Mitigate Cisplatin Activated B-Raf Signaling Cascade

As shown in FIGS. 5A and 5B, B-Raf, ERK, and MEK become phosphorylated and activated in HEL-OC1 cells upon 50 μM cisplatin treatment in a time-dependent manner. Dabrafenib treatment mitigates cisplatin-mediated 50 M, 1 hour activation of B-RAF, ERK and MEK in HEL-OC1 cells in a does dependent manner.

### Example 3 DABRAFENIB is Protective Against Cisplatin-Induced Hearing Loss in Adult Mice when Delivered Orally In Vivo

FIG. 6A shows a schedule of administration of dabrafenib and cisplatin to adult FVB mice (males and females). FIG. 6B shows reduced ABR threshold shifts of 11.8-15.0 dB on average were recorded on day 21 after first day of cisplatin and dabrafenib co-treatment, mean±SEM, *, P<0.05, compared to cisplatin alone by two-way ANOVA followed by a Bonferroni comparison.

### Example 4 DABRAFENIB is Protective Against Noise-Induced Hearing Loss in Adult Mice when Delivered Orally In Vivo

FIG. 7A shows the schedule of administration of dabrafenib and noise exposure to FVB mice (males and females). FIG. 7B shows reduced ABR threshold shifts of 18.1-21.9 dB on average were recorded on day 14 after first day of dabrafenib and noise exposure, mean±SEM, **, P<0.01, ***, P<0.001, compared to carrier by two-way ANOVA followed by a Bonferroni comparison.

Now referring to FIGS. 8A and 8B, Dabrafenib is protective against noise-induced hearing loss in adult mice when delivered orally forty-five minutes before the noise exposure. (A) Schedule of administration of dabrafenib and noise exposure to FVB mice (males and females). (B) Reduced ABR threshold shifts of 18.1-21.9 dB in average were recorded on day 14 after first day of dabrafenib and noise exposure, mean±SEM, **, P<0.01, ***, P<0.001, compared to carrier by two-way ANOVA followed by a Bonferroni comparison.

### Example 5: A Combination of Inhibitors Acts Synergistically

Now referring to FIG. 9, testing of a B-Raf/MEK1/2 inhibitor combination in mouse cochlear explant cultures. Compounds alone or combination of the compounds were added 1 h before cisplatin (150 μM) to P3 FVB cochlear explants for 24 h, and number of outer hair cells per 160 μm of middle turn regions of the cochlea were counted by phalloidin staining, mean±SEM, P=*<0.05, P=***<0.001, compared to cisplatin alone by unpaired two-tailed Student's t-test. The initial molar ratio between the compounds tested was determined by the ratio given currently to cancer patients (dabrafenib at 150 mg twice daily plus trametinib at 2 mg once daily).

### Example 6 Protective Effects are Shown in Zebrafish Lateral Line Neuromasts In Vivo

Methods of using a zebrafish model system to evaluate small molecules capable of decreasing, inhibiting, or preventing sensory hair cell damage or death are provided. Zebrafish are an advantageous animal model system for studying causes and prevention of hearing loss in comparison to mammalian animal model systems. The relative inaccessibility of hair cells in mammalian organisms limits their use as a high throughput model for identifying compounds that would prevent toxin mediated and other forms of hair cell death from occurring. The lateral line neuromast hair cells of zebrafish (Danio rerio) are structurally and functionally similar to mammalian sensory hair cells. Compounds SNS-314 (FIG. 10A) and crenolanib (FIG. 10B) protected in the cochlear explant culture assay against cisplatin-induced hair cell death. Dose-response of compounds SNS-314 and crenolanib in mouse cochlear explants treated with or without cisplatin. Compounds alone or compounds added 1 h before cisplatin (150 μM) to P3 FVB cochlear explants for 24 h, and number of outer hair cells per 160 μm of middle turn regions of the cochlea were counted by phalloidin staining, mean±SEM, P=*<0.05, P=**<0.005, *** P<0.0005 compared to cisplatin alone by unpaired two-tailed Student's t-test.

Protection of zebrafish lateral line neuromast hair cells in vivo. Five days post-fertilization Tg(bm3c: GFP) larvae were incubated with vehicle alone (DMSO), 400 μM of cisplatin (CP) for six hours or pre-treated with one of the compounds: dabrafenib (FIG. 11A), mubritinib (FIG. 11B), crenolanib (FIG. 11C) or SNS-314 (FIG. 11D) for 1 hour followed by a six hours co-incubation with the compound tested and CP 400 μM. After the treatment, animals were transferred to fresh fish water to recover for 1 hour and then fixed and immunostained for GFP and otoferlin. Quantification of the number of hair cells per neuromast after the different treatments represented as mean+/−SEM. Student's t test was performed, *p<0.05, ** p<0.01, *** p<0.001, compared versus CP 400 μM. Neuromasts inspected: SO3 (supraorbital line neuromast) and O1-2 (otic line neuromasts) from at least 3 different animals. Example 7 Synergistic effect of two inhibitors

FIG. 12 shows the compounds Dabrafenib (a B-Raf kinase inhibitor) and AZD5438 (a CDK2 kinase inhibitor) protects against cisplatin and noise ototoxicity, better than individual inhibitor, in mouse cochlear explants and mice in vivo. B-Raf/CDK2 inhibitor combination protects cisplatin induced hair cell loss in mouse cochlear explants. Compounds alone (purple bar) or combination of the compounds were added 1 h before cisplatin (150 μM) to P3 FVB cochlear explants for 24 hrs, and number of outer hair cells per 160 μm of middle turn regions of the cochlea were counted by phalloidin staining, mean±SEM, P=<0.05, P=**<0.005, compared to cisplatin alone by unpaired two-tailed Student's t-test. The initial AZD5438/dabrafenib combination tested was in the same molar ratio (0.34/30).

FIGS. 13A-13D show oral delivery of the combination of inhibitors provides protection effects that are significantly better than the use of individual compound. B-Raf/CDK2 inhibitor combination protects fully against noise induced hearing loss in mice when delivered orally. In this example, compounds for oral delivery were dissolved in the carrier 10% DMSO, 40% PEG300, 5% Tween-80 and 45% saline (0.9% NaCl) and were given in a volume of 10 ml/kg. Schedules of drugs and noise levels are shown in FIG. 13A. In FIG. 13B Dabrafenib (2×60 mg/kg/day) is given by oral delivery continuously for 3 days post-noise. In FIG. 13C AZD5438 (2×35 mg/kg/day) is given by oral delivery continuously for 3 days post-noise. In FIG. 13D complete protection against noise is achieved with combination of the two drugs for 3 days post-noise (dabrafenib (2×60 mg/kg/day) and AZD5438 (2×35 mg/kg/day), mean±SEM, P<0.05, P<0.01, compared to carrier alone by unpaired two-tailed Student's t-test

### Example 7

Experimental summary: To conclude, dabrafenib is a therapeutic candidate for preventing cisplatin-induced hearing loss. It has a low effective dose of one tenth of the human equivalent dose (3 mg/kg administered twice day), a good toxicity profile, a therapeutic index of at least 25 in the multi-dose cisplatin regimen, protects both female and male mice, reduces hearing loss in two different strains of mice (FVB/NJ and CBA/CaJ), offers protection from weight loss that occurs during cisplatin chemotherapy, and persistence of hearing protection for at least four months after cisplatin treatments.

### Example 8

Mouse Models For the cisplatin studies, 8-week-old CBA/CAJ mice were purchased from Jackson Laboratory (Bar Harbor, Maine, USA). Mice were then allowed to acclimate to the Animal Resource Facilities (ARF) at Creighton University before any experimental procedures began. For the noise studies, FVB/NJ breeding mice were purchased from Jackson Laboratory, bred in the ARF at Creighton University, and experiments were performed on the offspring of the breeders when mice were 8-10 weeks old. For pre- and post-experimental hearing tests, mice were anesthetized by Avertin (2,2,2-tribromoethanol) via intraperitoneal injection at a dose of 500 mg/kg, and complete anesthetization was determined via toe pinch. For all experiments, mice were randomly assigned to experimental groups with equal numbers of male and females.

### HEI-OC1 Cell Line and Collection of Cell Lysates

The HEI-OC1 cell line was maintained with DMEM 1× with glucose (4.5 g/liter), I-glutamine, and sodium pyruvate (12430-054, Gibco) with 10% FBS and ampicillin (50 μg/ml) added to the media. Cells were cultured under the conditions of 33° C. and 10% CO₂ while being passaged with 0.05% trypsin/EDTA. For the collection of cell lysates, HEI-OC1 cells were grown in T-75 flasks and allowed to grow to 80-90% confluency. 6 separate flasks of cells were used with each flask being its own treatment group. The 6 treatment groups were medium alone, 1 μM trametinib alone, 50 μM cisplatin alone, 50 μM cisplatin+0.01 μM trametinib, 50 μM cisplatin+0.1 μM trametinib, and 50 μM cisplatin+1 μM trametinib. HEI-OC1 cells were pre-treated with trametinib for 1 hour which was followed with cisplatin treatment for 1 hour. After one hour of cisplatin treatment, cell lysates were collected by dumping the media, putting cold DPBS in each flask, and scrapping the bottom of the flask with a cell scrapper to collect the cells in the DPBS. This was performed for a total of 4 separate times for each experimental group. Cells were then centrifuged at 2000 RPM for 5 minutes at 4° C. to form the cell pellet. Lysis buffer was then added to the cell pellet, cells were resuspended in the lysis buffer, and centrifuged for 20 minutes at 16,000 g at 4° C. The supernatant was then collected and put in −80° C. for storage before western blots were performed.

### Cancer Cell Lines and Cell-Titer Glo Assay

For tumor cell lines viability experiments, two neuroblastoma (SK-N-AS and SH-SY5Y) and a non-small cell lung carcinoma cell line (A549) were utilized. 9600 cells per well were plated in 6-replicates in 96 well plates and allowed to attach overnight at 37° C. in 5% CO₂. The following day, the tumor cell lines were pretreated with a range of 30 μM-4.57 nM of trametinib with 1/3 serial dilutions starting at 30 μM to 4.57 nM. The cells were then treated with cisplatin and incubated for 48 hours. The cisplatin concentration was dependent on the cell line and the IC₅₀ of cisplatin for the respective cell line was used as previously determined. The A549 cell line was treated with 25 μM cisplatin, SK-N-AS was 6.5 μM, and SH-SY5Y was 2 μM. Cell viability was then measured using the Cell Titer-Glo (Promega) assay. Medium alone-, cisplatin alone-, and drug alone were used as controls and the percentage viability was calculated as the viability compared to the medium alone treated cells. Drug plus cisplatin treated cells were compared to cisplatin alone treated cells to determine whether trametinib interfered with cisplatin's tumor killing ability.

Immunoblotting, Cell lysates that were stored in the −80° C. were thawed, and protein concentrations were determined with the BCA protein assay kit (23235, Thermo Fisher Scientific). Thirty micrograms of total cell lysate were loaded on 10% SDS-polyacrylamide gel electrophoresis gels. After running the gel and transferring to a nitrocellulose membrane, the following antibodies were used for immunoblotting analysis: anti-ERK1/2 (4695, Cell Signaling Technologies, 1:1000) anti-pERK1/2 (Thr²⁰²/Tyr²⁰⁴, 9101S, Cell Signaling Technologies, 1:1000) and anti-GAPDH (ab181602, abcam, 1:5000). Anti-rabbit (P0448) secondary antibody was purchased from Dako Laboratories and diluted 1:4000. National Institutes of Health (NIH) ImageJ software was used to quantify the band intensities and recorded as a ratio to the loading control GAPDH.

Multi-cycle cisplatin treatment model. Pre-experimental ABR were performed on 9-week-old CBA/CaJ mice with DPAOE performed when mice were 10 weeks old. Once mice were 12 weeks old, the 6-week cisplatin and trametinib treatment regimen began. Trametinib (1, 0.2, or 0.1 mg/kg) was administered via oral gavage 1 hour before 3 mg/kg cisplatin was administered to mice via intraperitoneal injection in the morning. Mice were then treated with trametinib or carrier again in the evening. Mice were treated with cisplatin once a day for 4 days and trametinib twice a day for 5 days with a 9-day recovery period in which no drugs were administered to the mice. This cycle was repeated two more times for a total of 3 cycles. Mice were treated with 3 mg/kg cisplatin for a total of 12 days (4 days per cycle with 3 cycles) and trametinib for a total of 15 days (5 days per cycle with 3 cycles). Immediately after the completion of cycle 3 (42 days after the first cisplatin injection), post-experimental ABR were performed with DPOAE performed one week after ABR. Cochleae were when harvested and put in 4% PFA. Cisplatin treated mice were injected by subcutaneous injection twice a day with 1 mL of warm saline to ameliorate dehydration. This continued until my body weight started to recover. Mouse weight was analyzed everyday throughout the 42-day treatment protocol. The cages of cisplatin-treated mice were placed on heating pads throughout the duration of the experiment until mice began to recover after the 3rd treatment cycle of the protocol. Food pellets dipped in DietGel Boost were placed on the cage floor of cisplatin-treated mice. The investigators and veterinary staff carefully monitored for changes in overall health and activity that may have resulted from cisplatin treatment.

Auditory Brainstem Response. ABR waveforms in anesthetized mice were recorded in a sound booth by using subdermal needles positioned in the skull, below the pinna, and at the base of the tail, and the responses were fed into a low-impedance Medusa digital biological amplifier system (RA4L; TDT; 20-dB gain). Mice were anesthetized by 500 mg/kg Avertin (2,2,2-Tribromoethanal, T4, 840-2; Sigma-Aldrich) with full anesthesia determined via toe pinch. At the tested frequencies (8, 16, and 32 kHz), the stimulus intensity was reduced in 10-dB steps from 90 to 10 dB to determine the hearing threshold. ABR waveforms were averaged in response to 500 tone bursts with the recorded signals filtered by a band-pass filter from 300 Hz to 3 kHz. ABR threshold was determined by the presence of at least 3 of the 5 waveform peaks. For noise exposure experiments, baseline ABR recordings were performed when mice were 7-8 weeks old and post experimental recordings were performed 14 days after noise exposure. For cisplatin experiments, ABR recordings were performed when mice were 9 weeks old and post experimental recordings were performed after the completion of the 42-day treatment protocol when mice were 18 weeks old. All beginning threshold values were between 10 and 40 dB at all tested frequencies. All thresholds were determined independently by two-three experimenters for each mouse who were blind to the treatment the mice received. Threshold shifts were calculated by subtracting the pre-noise exposure recording from the post-noise exposure recording. ABR wave one amplitudes were measured as the difference between the peak of wave 1 and the noise floor of the ABR trace.

Distortion Product Otoacoustic Emission, Distortion product otoacoustic emissions were recorded in a sound booth while mice were anesthetized. Mice were anesthetized by 500 mg/kg Avertin (2,2,2-Tribromoethanal, T4, 840-2; Sigma-Aldrich) with full anesthesia determined via toe pinch. DPOAE measurements were recorded using the TDT RZ6 processor and BioSigTZ software. The ER10B+ microphone system was inserted into the ear canal in a way that allowed for the path to the tympanic membrane to be unobstructed. DPOAE measurements occurred at 8, 12, 16, 24, and 32 kHz with an f2/f1 ratio of 1.2. Tone 1 was .909 of the center frequency and tone 2 was 1.09 of the center frequency. DPOAE data was recorded every 20.97 milliseconds and averaged 512 times at each intensity level and frequency. At each tested frequency, the stimulus intensity was reduced in 10 dB steps starting at 90 dB and ending at 10 dB. DPOAE threshold was determined by the presence of an emission above the noise floor. For noise exposure experiments, baseline DPOAE recordings were performed when mice were 7-8 weeks old and post experimental recordings occurred after 14 days following noise exposure. For cisplatin experiments, ABR recordings were performed when mice were 10 weeks old and post experimental recordings were performed after the completion of the 42-day treatment protocol when mice were 19 weeks old. Threshold shifts were calculated by subtracting the pre-noise exposure recording from the post-noise exposure recording.

Noise Exposure. Mice were placed in individual cages in a custom-made wire container. System RZ6 (TDT) equipment produced the sound stimulus which was amplified using a 75-A power amplifier (Crown). A JBL speaker delivered the sound to the mice in the individual chambers. The sound pressure level was calibrated using an NSRT-mk3 (convergence instruments) microphone and all chambers were within 0.5 dB of each other to ensure equal noise exposure. Mice were exposed to 100 dB SPL noise for 2 hours with an octave band noise of 8 to 16 kHz.

Tissue preparation, immunofluorescence, and OHCs counts. For OHC counts, cochleae from adult mice were prepared and examined as described previously [24,25,64]. Cochleae were placed in 4% PFA following harvesting. Cochlear samples were stained with anti-myosin VI (1:400; 25-6791, Proteus Bioscience) with the secondary antibody purchased from Invitrogen coupled to anti-rabbit Alexa Fluor 488 (1:400; A11034). All images were acquired with a confocal microscope (LSM 700 or 710, Zeiss). Outer hair cell counts were determined by the total amount of outer hair cells in a 160 μm region. Counts were determined for the 8, 16, and 32 kHz regions. Cochleae from each experimental group were randomly selected to be imaged for outer hair cell counts.

For ctbp2 counts, cochlea was harvested and prepared the same as cochlea for OHC counts. Organs of Corti were micro dissected and co-stained with anti-Ctbp2 (1:800; 612044, BD Transduction) and myosin VI (1:400; 25-6791, Proteus Biosciences). Goat anti-rabbit Alexa Fluor 488 (1:400; A11034) and goat anti-mouse Alexa Fluor 647 (1:800; A32728) were purchased from Invitrogen as secondary antibodies. Confocal Imaging was performed using a Zeiss 700 upright scanning confocal microscope with images taken with the 63× objective lens. Final images were achieved by taking a z stack image of the organ of Corti and processed through the ZenBlack program. The number of Ctbp2 puncta were counted per IHC with a total of 12-14 IHCs analyzed at the 16 kHz region. The total amount of Ctbp2 puncta were divided by the total amount of IHCs in the 16 kHz region to determine the number of Ctbp2 puncta per IHC.

Statistical Analysis. Statistics was performed using Prism (GraphPad Software). Two-way analysis of variance (ANOVA) or One-way ANOVA with Bonferroni post hoc test was used to determine mean difference and statistical significance. Specific statistical tests used for each experiment are in the figure legends. Statistical significance was determined when P<0.05.

Now referring to FIG. 14A, a schematic of the MAPK phosphorylation cascade in which dabrafenib inhibits BRAF, trametinib inhibits MEK1/2, and tizaterkib inhibits ERK1/2 is shown. Now referring to FIGS. 14B and 14C, western blots using HEI-OC1 lysates that were treated with cisplatin and different concentrations of trametinib. Six different treatments were performed a total of four separate times and four individual western blots were run. The six treatment groups are as follows: medium alone, 1 μM trametinib alone, 50 μM cisplatin alone, 50 μM cisplatin co-administered with 0.01 μM trametinib, 50 μM cisplatin co-administered 0.1 μM trametinib, and 50 μM cisplatin co-administered 1 μM trametinib.

Phosphorylated ERK1/2 (PERK) was chosen as the protein of interest because ERK1/2 is directly downstream of MEK1/2 in the MAPK pathway and trametinib does not inhibit MEK1/2 phosphorylation, but it prevents MEK1/2 from having the catalytic ability to activate downstream proteins. GAPDH was used as the loading control and the band intensity of pERK was divided by the intensity of GAPDH to get the normalized ratio. All groups were then normalized to the medium alone treatment group. A dose of 50 μM cisplatin significantly increased pERK in the HEI-OC1 cell line compared to medium alone and all concentrations of trametinib decreased pERK. There was over a threefold decrease in ERK1/2 phosphorylation following co-administration of 0.01 μM trametinib and cisplatin compared to the cisplatin alone treated HEI-OC1 cells. Concentrations of 0.1 and 1 μM trametinib completely abrogated ERK1/2 phosphorylation in the presence or absence of cisplatin.

Now referring to FIGS. 14D and 14F, trametinib was then treated with cisplatin in several different cancer cell lines that cisplatin is commonly used for treatment of the respective tumors (neuroblastoma and lung carcinoma. The CellTiter-Glo Assay was performed to measure the cell viability and determine whether trametinib interferes with cisplatin's tumor killing ability in vitro. In a 96 well plate, 9600 cells were plated into each well and the medium alone wells were considered 100% cell viability. Cisplatin concentration decreased cell viability by approximately 50% and all wells treated with cisplatin had the same cisplatin concentration. For the wells treated with trametinib, cells were treated with the drug by itself or combined with cisplatin. A wide range of doses were utilized starting at 30 μM and serial dilutions of 1/3 were performed until a low dose of 4.57 nM was achieved to show that neither high nor low concentrations of trametinib interfere with cisplatin. Cells co-treated with cisplatin and trametinib were compared to cisplatin alone treated wells to determine whether any interference occurred between the two drugs. Each treatment group had 6 wells. For all three cell lines that were treated with both drugs, SK-N-AS (neuroblastoma), SH-SY5Y (neuroblastoma), and A549 (small-cell lung carcinoma), trametinib did not interfere with cisplatin's tumor killing ability at any of the tested doses.

Now referring to FIGS. D-F, trametinib and cisplatin treated cells had less cell viability than the cisplatin alone cells, which indicates that trametinib treatment with cisplatin enhances the killing of the tumor cells compared to cisplatin alone. Trametinib by itself reduced cell viability in all three cancer cell lines, especially at concentrations of 123 nM and higher.

Now referring to FIGS. 15A-15D, to determine whether oral administration of trametinib protects from cisplatin-induced hearing loss, a previously optimized, clinically relevant mouse model of cisplatin administration was used. Fernandez et al. Clinically Relevant Mouse Model of Cisplatin-induced Ototoxicity, Har Res 2019; 375, 66-74. Mice were treated with 3 mg/kg cisplatin in the morning and treated with trametinib 45 minutes before cisplatin in the morning and again in the evening. There was a total of four days of cisplatin treatment and five days of trametinib treatment which was followed up with a nine-day recovery period in which no drugs were administered to the mice. This cycle was repeated for a total of three times and hearing tests were performed before and after treatment to determine the amount of hearing loss per experimental cohort.

Mice co-treated with 1 mg/kg trametinib, and cisplatin had significantly lower ABR threshold shifts at 8, 16, and 32 kHz with average threshold shift reductions of 35, 40, and 41 dB compared to the cisplatin alone treated mice, respectively. Mice co-treated with 0.2 mg/kg trametinib and cisplatin had significantly lower ABR threshold shifts of 22 dB at 16 KHz and 24 dB at 32 kHz compared to cisplatin alone treated mice. 0.1 mg.kg trametinib did not significantly decrease ABR threshold shifts compared to cisplatin alone treated mice. 1 mg/kg trametinib co-treated mice with cisplatin had significantly higher ABR wave 1 amplitude at the 16 kHz region compared to cisplatin alone treated mice at 90-, 80-, 70-, and 60 dB while the mice co-treated with 0.2 mg/kg trametinib and cisplatin had significantly higher wave 1 amplitudes at 90- and 80 dB. DPOAE threshold shifts were also measured and 1 mg/kg trametinib co-treated mice had significantly lower DPOAE threshold shifts at 12 and 16 kHz compared to the cisplatin alone treatment while 0.2 mg/kg trametinib co-treated mice with cisplatin had significantly lower DPOAE threshold shifts at 12 KHz. Cisplatin alone treated mice had an average DPOAE threshold shift of 40 dB±4 and 43 dB±4 at the 12 and 16 kHz, respectively, while mice co-treated with 1 mg/kg trametinib, and cisplatin had an average DPOAE threshold shift of 12 dB±4 at 12 kHz and 8 dB±6 at 16 KHz. Mice co-treated with 0.2 mg/kg trametinib and cisplatin had an average DPOAE threshold shift of 24 dB±4 at 12 KHz.

Now referring to FIG. 16, Following the post experiment hearing tests, mouse cochleae were collected, and dissections were performed and whole mount images were stained with myosin VI to measure the number of OHC's in each treatment group. Carrier alone treated mice had an average of 65, 64, and 64 OHCs per 160 μm in the apical, middle, and basal regions respectively while cisplatin alone treated mice had an average of 56 OHCs at the apical region, 32 at the middle region, and 4 at the basal region. Mice treated with 1 mg/kg trametinib alone had the same number of OHCs compared to the carrier alone cohort at all cochlear regions. Mice co-treated with 1 mg/kg trametinib or 0.2 mg/kg trametinib with cisplatin had significantly more OHCs at the middle and basal regions compared to cisplatin alone treated mice. Mice co-treated with 1 mg/kg trametinib, and cisplatin had an average of 59, 56, and 43 OHCs per 160 μm at the apical, middle and basal regions, respectively, while mice co-treated with 0.2 mg/kg trametinib and cisplatin had an average of 55, 50, and 26 OHCs per 160 μm at the apical, middle, and basal regions, respectively. 1 mg/kg trametinib plus cisplatin treated mice had a statistically significant higher average of 18 OHCs more per 160 μm at the basal region compared to mice treated with 0.2 mg/kg trametinib and cisplatin.

Now referring to FIG. 17, Mice were weighed everyday throughout the 42-day treatment protocol shown in FIG. 15A. Mice co-treated with both doses of trametinib, and cisplatin had significantly less weight loss at days 26, 28, 29, 31, and 39-42 compared to the cisplatin alone treated mice. Cisplatin alone treated mice had a maximum average of 28% weight loss compared to baseline body weight and both trametinib co-treated groups had a maximum average of 22% weight loss throughout the treatment protocol. Trametinib treatment in the absence of cisplatin did not cause any weight loss and mice treated with trametinib alone gradually gained weight throughout the protocol just like carrier alone mice. The 1 mg/kg trametinib co-treated group with cisplatin had significant mouse death with only 36% of mice surviving to the end of treatment protocol while 78% of cisplatin alone treated mice lived to the end of the treatment protocol. 92% of the 0.2 mg/kg co-treated mice with cisplatin survived throughout the entire protocol.

Now referring to FIG. 18, to determine whether trametinib protects from noise-induced hearing loss in vivo, a model of NIHL was used that is performed in FVB mice. Briefly, mice were exposed to 100 dB SPL for 2 hours at 8-16 kHz, which induces permanent threshold shifts in FVB mice, and treatment with 3.15 mg/kg trametinib began 24 hours following the noise exposure. Mice were treated twice a day for three days, once in the morning and once in the evening. Hearing tests were performed before and after the treatment protocol to determine how much hearing loss occurred for each treatment group. FVB mice treated with trametinib had significantly lower ABR threshold shifts compared to the noise alone mice. Trametinib treated mice following noise exposure had an average ABR threshold shift decrease of 19 dB at 8 KHz and 18 dB at 16 kHz compared to noise alone mice. Oral administration of trametinib without noise exposure did not induce any hearing loss in the FVB mice. After post-experimental hearing tests were performed, the cochleae of the mice were harvested and the organ of Corti was dissected to measure ribbon synapse degeneration that is observed following this noise exposure protocol. Outer hair cell loss is not observed in this mouse model of permanent hearing loss, but synaptic damage commonly occurs. Myosin VI stained for hair cells and ctbp2 stained the presynaptic ribbon synapses. The number of ctbp2 puncta per IHC at the 16 kHz region were quantified and trametinib treated mice following noise exposure have significantly more ctbp2 puncta per IHC compared to noise alone mice. Noise+trametinib treated mice have an average of 8.7±0.4 ctbp2 puncta per IHC and noise alone mice have an average of 6.4±0.6. Trametinib alone treated mice have an average of 13.4±0.7 ctbp2 puncta per IHC which is comparable to carrier alone treated FVB mice,

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A method to prevent ototoxicity comprising orally administering to subject in need thereof: a sufficient amount of a RAF inhibitor to prevent hearing loss, a sufficient amount of a ERK1/2 inhibitor to prevent hearing loss and a sufficient amount of a MEK1/2 inhibitor to prevent hearing loss.

2. A method to prevent cisplatin-induced hearing loss comprising orally administering to subject in need thereof a pharmaceutical composition comprised of a sufficient amount of trametinib to prevent hearing loss due to cisplatin treatment, wherein the sufficient amount of trametinib, wherein the sufficient amount of trametinib ranges from 0.01 μM to 1.0 μM of trametinib.

3. The method of claim 2, further comprising the step of orally administering to subject in need thereof after a first rest period a pharmaceutical composition comprised of: a sufficient amount of trametinib to prevent hearing loss due to cisplatin treatment, wherein the sufficient amount of trametinib, wherein the sufficient amount of trametinib ranges from 0.01 μM to 1.0 μM of trametinib.

4. The method of claim 3, further comprising the step of orally administering to subject in need thereof after a second rest period a pharmaceutical composition comprised of: a sufficient amount of trametinib to prevent hearing loss due to cisplatin treatment, wherein the sufficient amount of trametinib, wherein the sufficient amount of trametinib ranges from 0.01 μM to 1.0 μM of trametinib

5. A pharmaceutical composition in a unit dose form comprising cisplatin and an effective amount of a MEK1/2 inhibitor to prevent hearing loss due to ototoxicity, wherein the MEK inhibitor is trametinib.

6. The pharmaceutical composition of claim 5 further comprised of: a sufficient amount of RAF inhibitor to prevent hearing loss due to ototoxicity.

7. The pharmaceutical composition of claim 5 further comprised of: a sufficient amount of ERK inhibitor to prevent hearing loss due to ototoxicity.
